(19) United States
(12) Patent Application Publication
(10) Pub. No.: US 2001/0025030 A1
(43) Pub. Date: Sep. 27, 2001

(54) cDNA for human methylenetetrahydrofolate reductase and uses thereof

(76) Inventors: Rima Rozen, Montreal West (CA); Jaspreet Sekhon, Vancouver (CA)

Correspondence Address: Karen L. Elbing, Ph.D., Clark & Elbing LLP, 176 Federal Street, Boston, MA, 02110, US

(21) Appl. No.: 09/728,910

(22) Filed: Dec. 1, 2000

Related U.S. Application Data
(63) Continuation-in-part of application No. 09/258,928, filed on Mar. 1, 1999, now Pat. No. 6,218,120.

(51) Int. Cl. A61K 48/00; C12Q 1/68

(52) U.S. Cl. 514/44; 435/6

(57) ABSTRACT

The invention features antisense molecules that inhibit the expression of MTHFR. These compounds are useful in the treatment, stabilization, and prevention of various cancers.

### RELATED APPLICATIONS

This application is a continuation-in-part of U.S. patent application Ser. No. 09/258,928 filed, Mar. 1, 1999.

### BACKGROUND OF THE INVENTION

(a) Field of the Invention

The invention relates to a cDNA probe for human methylenetetrahydrofolate reductase (MTHFR), and its uses.

(b) Description of Prior Art

Folic acid derivatives are coenzymes for several critical single-carbon transfer reactions, including reactions in the biosynthesis of purines, thymidylate and methionine. Methylenetetrahydrofolate reductase (MTHFR; EC 1.5.1.20) catalyses the NADPH-linked reduction of 5,10-methylenetetrahydrofolate to 5-methyltetrahydrofolate, a co-substrate for methylation of homocysteine to methionine. The porcine liver enzyme, a flavoprotein, has been purified to homogeneity; it is a homodimer of 77-kDa subunits. Partial proteolysis of the porcine peptide has revealed two spatially distinct domains: an N-terminal domain of 40 kDa and a C-terminal domain of 37 kDa. The latter domain contains the binding site for the allosteric regulator S-adenosylmethionine.

Hereditary deficiency of MTHFR, an autosomal recessive disorder, is the most common inborn error of folic acid metabolism. A block in the production of methyltetrahydrofolate leads to elevated homocysteine with low to normal levels of methionine. Patients with severe deficiencies of MTHFR (0-20% activity in fibroblasts) can have variable phenotypes. Developmental delay, mental retardation, motor and gait abnormalities, peripheral neuropathy, seizures and psychiatric disturbances have been reported in this group, although at least one patient with severe MTHFR deficiency was asymptomatic. Pathologic changes in the severe form include the vascular changes that have been found in other conditions with elevated homocysteine, as well as reduced neurotransmitter and methionine levels in the CNS. A milder deficiency of MTHFR (35-50% activity) has been described in patients with coronary artery disease (see below). Genetic heterogeneity is likely, considering the diverse clinical features, the variable levels of enzyme activity, and the differential heat inactivation profiles of the reductase in patients' cells.

Coronary artery disease (CAD) accounts for 25% of deaths of Canadians. Cardiovascular risk factors (male sex, family history, smoking, hypertension, dyslipoproteinemia and diabetes) account for approximately 60 to 70% of the ability to discriminate CAD patients from healthy subjects. Elevated plasma homocysteine has also been shown to be an independent risk factor for cardiovascular disease.

Homocysteine is a sulfhydryl-containing amino acid that is formed by the demethylation of methionine. It is normally metabolized to cysteine (transsulfuration) or re-methylated to methionine. Inborn errors of metabolism (as in severe MTHFR deficiency) causing extreme elevations of homocysteine in plasma, with homocystinuria, are associated with premature vascular disease and widespread arterial and venous thrombotic phenomena. Milder elevations of plasma homocysteine (as in mild MTHFR deficiency) have been associated with the development of peripheral vascular disease, cerebrovascular disease and premature CAD.

Homocysteine remethylation to methionine requires the folic acid intermediate, 5-methyltetrahydrofolate, which is produced from 5,10-methylenetetrahydrofolate folate through the action of 5,10-methylenetetrahydrofolate reductase (MTHFR). Deficiency of MTHFR results in an inability to metabolize homocysteine to methionine; elevated plasma homocysteine and decreased methionine are the metabolic consequences of the block. Severe deficiencies of MTHFR (less than 20% of activity of controls) as described above, are associated with early-onset neurologic symptoms (mental retardation, peripheral neuropathy, seizures, etc.) and with atherosclerotic changes and thromboembolism. Milder deficiencies of MTHFR (35-50% of activity of controls), with a thermolabile form of the enzyme, are seen in patients with cardiovascular disease without obvious neurologic abnormalities.

In a survey of 212 patients with proven coronary artery disease, the thermolabile form of MTHFR was found in 17% of the CAD group and 5% of controls. In a subsequent report on 339 subjects who underwent coronary angiography, a correlation was found between thermolabile MTHFR and the degree of coronary artery stenosis. Again, traditional risk factors (age, sex, smoking, hypertension, etc.) were not significantly associated with thermolabile MTHFR. All the studies on MTHFR were performed by enzymatic assays of MTHFR in lymphocytes, with measurements of activity before and after heat treatment to determine thermolability of the enzyme.

Since 5-methyltetrahydrofolate, the product of the MTHFR reaction, is the primary form of circulatory folate, a deficiency in MTHFR might lead to other types of disorders. For example, periconceptual folate administration to women reduces the occurrence and recurrence of neural tube defects in their offspring. Neural tube defects are a group of developmental malformations (meningomyelocele, anencephaly, and encephalocele) that arise due to failure of closure of the neural tube. Elevated levels of plasma homocysteine have been reported in mothers of children with neural tube defects. The elevated plasma homocysteine could be due to a deficiency of MTHFR, as described above for cardiovascular disease.

Neuroblastomas are tumors derived from neural crest cells. Many of these tumors have been reported to have deletions of human chromosome region 1p36, the region of the genome to which MTHFR has been mapped. It is possible that MTHFR deletions/mutations are responsible for or contribute to the formation of this type of tumor. MTHFR abnormalities may also contribution to the formation of other types of tumors, such as colorectal tumors, since high dietary folate has been shown to be inversely associated with risk of colorectal carcinomas.

MTHFR activity is required for homocysteine methylation to methionine. Methionine is necessary for the formation of S-adenosylmethionine, the primary methyl donor for methylation of DNA, proteins, lipids, neurotransmitters, etc. Abnormalities in MTHFR might lead to lower levels of methionine and S-adenosylmethionine, as well as to elevated homocysteine. Disruption of methylation processes could result in a wide variety of conditions, such as neoplasias, developmental anomalies, neurologic disorders, etc.

Although the MTHFR gene in Escherichia coli (metF) has been isolated and sequenced, molecular studies of the enzyme in higher organisms have been limited without the availability of an eukaryotic cDNA.

It would be highly desirable to be provided with a cDNA probe for human methylenetetrahydrofolate reductase (MTHFR). This probe would be used for identification of sequence abnormalities in individuals with severe or mild MTHFR deficiency, including cardiovascular patients and patients with neurologic symptoms or tumors. The probe would also be used in gene therapy, isolation of the gene, and expression studies to produce the MTHFR protein. The probe would also provide the amino acid sequence of the human MTHFR protein, which would be useful for therapy of MTHFR deficiency by biochemical or pharmacological approaches.

It would be highly desirable to be provided with a molecular description of mutations in methylenetetrahydrofolate reductase deficiency.

Patients with sequence abnormalities in MTHFR might have different responses to drugs, possibly but not limited to drugs that affect folate metabolism. Therefore, it would be useful to know if these mutations are present before determining the appropriate therapy. The drugs/diseases for which this might be relevant include cancer chemotherapeutic agents, antibiotics, antiepileptic medication, antiarthritic medication, etc.

### SUMMARY OF THE INVENTION

One aim of the present invention is to provide a cDNA probe for human methylenetetrahydrofolate reductase (MTHFR).

Another aim of the present invention is to provide a molecular description of mutations in methylenetetrahydrofolate reductase deficiency.

Another aim of the present invention is to provide a nucleic acid and amino acid sequence for human methylenetetrahydrofolate reductase.

Another aim of the present invention is to provide potential therapy for individuals with methylenetetrahydrofolate reductase deficiency.

Another aim of the present invention is to provide a system for synthesis of MTHFR protein in vitro.

A further aim of the present invention is to provide technology/protocol for identification of sequence changes in the MTHFR gene.

In accordance with one aspect of the present invention, there is provided a cDNA probe for human methylenetetrahydrofolate reductase (MTHFR) gene encoded by a nucleotide sequence as set forth in SEQ ID NO:1 or having an amino acid sequence as set forth in SEQ ID NO:2. The probe comprises a nucleotide sequence that hybridizes to the MTHFR nucleotide sequence, or an amino acid sequence that hybridizes to the MTHFR amino acid sequence.

In accordance with another aspect of the present invention, there is provided a method of diagnosis of methylenetetrahydrofolate reductase (MTHFR) deficiency in a patient with MTHFR deficiency. The method comprises the steps of amplifying a DNA sample obtained from the patient or reverse-transcripting a RNA sample obtained from the patient into a DNA and amplifying the DNA, and analyzing the amplified DNA to determine at least one sequence abnormality with respect to a human MTHFR encoded by a nucleotide sequence as set forth in SEQ ID NO:1 or having an amino acid sequence as set forth in SEQ ID NO:2, the sequence abnormality being indicative of MTHFR deficiency.

The sequence abnormality may comprise a mutation selected from a group consisting of 167G/A, 482G/A, 559C/T, 677C/T, 692C/T, 764C/T, 792+1G/A, 985C/T, 1015C/T, 1081C/T, 1298A/C and 1317T/C.

The selected mutation may consist of 677C/T.

The MTHFR deficiency may be associated with a disease, disorder, or dsyfuntion selected from a group consisting of cardiovascular disorders, cancer, osteoporosis, increased risk of occurrence of a neural tube defect in an offspring of said patient, neurological disorders, and disorders influenced by folic acid metabolism.

The cancer may be selected from a group consisting of neuroblastomas and colorectal carcinomas.

The disorder may consist of osteoporosis.

In accordance with yet another aspect of the present invention, there is provided a method for gene therapy of methylenetetrahydrofolate reductase (MTHFR) deficiency in a patient. The method comprises the steps of producing a recombinant vector for expression of MTHFR under the control of a suitable promoter, the MTHFR being encoded by a nucleotide sequence as set forth in SEQ ID NO:1 or having an amino acid sequence as set forth in SEQ ID NO:2, and transfecting the patient with the vector for expression of MTHFR.

In accordance with yet another aspect of the present invention, there is provided a human methylenetetrahydrofolate reductase (MTHFR) protein encoded by a nucleotide sequence as set forth in SEQ ID NO:1 or having an amino acid sequence as set forth in SEQ ID NO:2.

In accordance with yet another aspect of the present invention, there is provided a recombinant human methylenetetrahydrofolate reductase (MTHFR) protein encoded by a nucleotide sequence as set forth in SEQ ID NO:1 or having an amino acid sequence as set forth in SEQ ID NO:2.

In accordance with yet another aspect of the present invention, there is provided a method of treatment of MTHFR-deficiency in a patient that comprises administering such a MTHFR protein.

The MTHFR deficiency may be associated with a cancer.

The cancer may be selected from a group consisting of neuroblastomas and colorectal carcinomas.

In accordance with yet another aspect of the present invention, there is provided a method of preventing an occurrence of a neural tube defect in an offspring of a patient. The method comprises administering to the patient such a MTHFR protein.

In accordance with yet another aspect of the present invention, there is provided a method for determining drug susceptibility, drug response or drug toxicity of a patient having a methylenetetrahydrofolate reductase (MTHFR) deficiency. The method comprises the steps of amplifying a DNA sample obtained from the patient or reverse-transcripting a RNA sample obtained from the patient into a DNA and amplifying said DNA, analyzing the amplified DNA to determine a sequence abnormality in a MTHFR sequence, the MTHFR sequence being encoded by a nucleotide sequence as set forth in SEQ ID NO:1 or having an amino acid sequence as set forth in SEQ ID NO:2, and administering the drug to the patient and determining the sequence abnormality associated with the patient susceptibility, response or toxicity to the drug.

The sequence abnormality may comprise a mutation selected from a group consisting of 167G/A, 482G/A, 559C/T, 677C/T, 692C/T, 764C/T, 792+1G/A, 985C/T, 1015C/T, 1081C/T, 1298A/C and 1317T/C and the drug may be selected from a group consisting of cancer chemotherapeutic agents, antibiotics, antiepileptic agents, and antiarthritic agents.

The MTHFR deficiency may be associated with a disorder selected from a group consisting of cardiovascular disorders, coronary and arterial disorders, neurological disorders, increased risk of occurrence of a neural tube defect in an offspring, cancer, osteoporosis and other disorders influenced by folic acid metabolism.

In accordance with yet another aspect of the present invention, there is provided a method of treatment of a patient having a cancer comprising the step of inhibiting gene expression for a MTHFR protein or a mRNA produced from the gene.

In accordance with yet another aspect of the present invention, there is provided a method of treatment of a patient having a cancer comprising the step of inhibiting the MTHFR protein.

In a related aspect, the invention provides a method of treating, stabilizing, or preventing cancer in a mammal that involves inhibiting MTHFR protein expression in the mammal. Preferably, this inhibition of MTHFR protein expression involves administering to, or expressing in, the mammal an antisense nucleic acid that binds an MTHFR nucleic acid. In one preferred embodiment, the antisense nucleic acid binds an exon of MTHFR, such as exon 5. In another preferred embodiment, the antisense nucleic acid is not allele-specific. In yet another preferred embodiment, the antisense nucleic acid is contained in a recombinant viral vector, which may be administered to a mammal for the in vivo expression of the antisense nucleic acid. Preferred routes of administration of an antisense molecule that inhibits MTHFR expression include intravenous, parenteral, subcutaneous, intramuscular, intraventricular, intraperitoneal, or any other route sufficient to provide a dose adequate to treat, stabilize, or prevent cancer. In another preferred embodiment, the compound is directly administered to a tumor. In preferred embodiments, the cancer is a breast carcinoma, colon carcinoma, colorectal carcinoma, neuroblastoma, or glioma. Preferably, the mammal is a human.

In another related aspect, the invention features a compound that inhibits MTHFR protein expression in a mammal (e.g., a human) by an amount sufficient to treat, stabilize, or prevent cancer in the mammal. Preferred compounds include antisense nucleic acids that bind an MTHFR nucleic acid. Preferably, the antisense nucleic acids bind an exon of MTHFR, such as exon 5. In one preferred embodiment, the antisense nucleic acid is not allele-specific. Examples of cancers that may be treated, stabilized, or prevented with a compound of the invention include breast carcinomas, colon carcinomas, colorectal carcinomas, neuroblastomas, and gliomas.

In still another related aspect, the invention features a vector encoding an antisense nucleic acid that binds an MTHFR nucleic acid and inhibits MTHFR protein expression. Such a vector is useful to treat, stabilize, or prevent cancer in a mammal. Preferably, the antisense nucleic acid is not allele-specific. Cancers that may be treated, stabilized, or prevented by the administration of the vector to a mammal include, but are not limited to, breast carcinomas, colon carcinomas, colorectal carcinomas, neuroblastomas, and gliomas. In one preferred embodiment, the vector is a viral vector, such as an adenoviral, adeno-associated viral, retroviral, lentiviral, baculovirus, or herpes viral vector.

As used herein, by “inhibiting MTHFR protein expression” is meant inhibiting MTHFR transcription or translation. The level of MTHFR mRNA may be determined by standard Northern blot analysis, and the level of MTHFR protein may be determined by standard Western blot analysis, such as the analyses described herein or those described by, for example, Ausubel et al. (Current Protocols in Molecular Biology, John Wiley & Sons, New York, 2000). In one preferred embodiment, the level of MTHFR protein is determined by measuring the level of MTHFR enzymatic activity, as described herein. Alternatively, a decrease in the level of MTHFR protein may be assessed by measuring the resulting increase in homocysteine or the decrease in 5-methyltetrahydrofolate or methionine, as described herein. Preferably, the level of MTHFR mRNA, protein, or enzymatic activity or the level of methionine in cells that have been administered a compound of the invention is at least 20, 40, 60, 80, 90, or 95% lower than the corresponding levels in the absence of the compound. Preferably, the level of homocysteine in the presence of the compound is at least 20, 40, 60, 80, 90, 100, 200, or 500% greater than in the absence of the compound. In another preferred embodiment, the level of MTHFR mRNA, protein, or enzymatic activity is equal to or less than 20, 20, 5, or 2-fold above the corresponding level in control cells that do not express functional MTHFR, such as cells homozygous for an MTHFR nonsense mutation.

By “antisense nucleic acid” is meant a sequence of two or more covalently bonded naturally-occurring or modified deoxyribonucleotides, ribonucleotides, or a combination of deoxyribonucleotides and ribonucleotides. The antisense nucleic acid may bind to any region of an MTHFR nucleic acid (i.e., an MTHFR deoxyribonucleic acid or ribonucleic acid). If desired, this binding may be confirmed using standard assays, such as gel-shift or hybridization assays (see, for example, Ausubel et al. (2000), supra). Preferred regions that are bound by the antisense nucleic acid include all or part of any one of exons 1-11 or any other MTHFR exon (Goyette et al., Mammalian Genome 9:652-656, 1998). Examples of other regions of MTHFR that may be bound by an antisense nucleic acid include all or part of a regulatory region of MTHFR such as a transcription factor binding site, the MTHFR promoter, an intron, a 5′ untranslated region (UTR), and a 3′ UTR (Goyette et al., Mammalian Genome 9:652-656, 1998; Homberger et al., Eur. J. Hum. Genet. 8:725-729, 2000; Gaughan et al., Gene 257:279-289, 200). Preferably, the antisense oligonucleotide is a phosphorothioate nucleic acid in which a non-bridging phosphoryl oxygen in one or more of the nucleotides is replaced with sulfur. Preferred antisense nucleic acids include the phosphorothioate oligonucleotide 5′-AGCTGCCGAAGGGAGTGGTA-3′ and the corresponding unmodified 5′-AGCTGCCGAAGGGAGTGGTA-3′ (SEQ ID NO: 15). Preferred nucleic acids contain between 10 and 20, 20 and 40, 40 and 60, 60 and 80, 80 and 100, 100 and 150, 150 and 250, or 250 and 350 nucleotides, inclusive. In another preferred embodiment, the nucleic acid contains between 15 and 25 or between 18 and 22 nucleotides, inclusive. Other preferred antisense nucleic acids include nucleic acids that have a polynucleotide sequence that is at least 60, 70, 80, 90, 95, or 100% identical to the reverse complement of a region of an MTHFR nucleic acid. Preferably, the polynucleotide sequence of the antisense nucleic acid is less than 50, 40, 30, 20, 10, 5, or 1% identical to the reverse complement of a region in another human expressed sequence (EST) or, more preferably, in any other reported human EST. Still other preferred antisense nucleic acids hybridize to an MTHFR nucleic acid under high stringency conditions. MTHFR antisense oligonucleotides may be tested in the assays described herein to determine whether they inhibit MTHFR protein expression or decrease the viability of cancerous cells. In addition, the antisense oligonucleotides may be tested in any standard animal model for cancer, such as the nude mouse model described herein.

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications.

By “high stringency conditions” is meant hybridization in 2X SSC at 40° C. For other definitions of high stringency conditions, see F. Ausubel et al., Current Protocols in Molecular Biology, pp. 6.3.1-6.3.6, John Wiley & Sons, New York, N.Y., 2000.

By “not allele-specific” or “non allele-specific” is meant capable of binding to at least two different MTHFR alleles. In one preferred embodiment, an MTHFR antisense nucleic acid that is not allele-specific binds both MTHFR alleles of a given mammal. In another preferred embodiment, the antisense nucleic acid binds at least 70, 80, 90, or 100% of the human MTHFR alleles. Preferably, the affinity of the antisense nucleic acid for each MTHFR allele in the mammal differs by less than 500, 200, 100, 50, 20, or 10%, as measured using standard gel-shift or hybridization assays (see, for example, Ausubel et al. (2000), supra). In another preferred embodiment, the antisense nucleic acid does not bind to a region of MTHFR that contains a polymorphic site.

By “treating, stabilizing, or preventing cancer” is meant causing a reduction in the size of a tumor, slowing or preventing an increase in the size of a tumor, increasing the disease-free survival time between the disappearance of a tumor and its reappearance, preventing an initial or subsequent occurrence of a tumor, or reducing an adverse symptom associated with a tumor. In one preferred embodiment, the percent of cancerous cells surviving the treatment is at least 20, 40, 60, 80, or 100% lower than the initial number of cancerous cells, as measured using any standard assay, such as those described herein. Preferably, the decrease in the number of cancerous cells induced by administration of a compound of the invention is at least 2, 5, 10, 20, or 50-fold greater than the decrease in the number of non-cancerous cells. In yet another preferred embodiment, the number of cancerous cells present after administration of an MTHFR antisense nucleotide is at least 2, 5, 10, 20, or 50-fold lower than the number of cancerous cells present after administration of a control nucleotide that has a polynucleotide sequence less than 70% identical to the reverse complement of a region of an MTHFR nucleic acid. Preferably, the methods of the present invention result in a decrease of 20, 40, 60, 80, or 100% in the size of a tumor as determined using standard methods. Preferably, at least 20, 40, 60, 80, 90, or 95% of the treated subjects have a complete remission in which all evidence of the cancer disappears. Preferably, the cancer does not reappear or reappears after at least 5, 10, 15, or 20 years.

A “polymorphism” is intended to mean a mutation or allelic variance present in 1% or more of alleles of the general population. A polymorphism is disease-causing when it is present in patients with a disease but not in the general population. However, a polymorphism present both in patients having a disease and in the general population is not necessarily benign. The definition of a disease-causing substitution, as distinct from a benign polymorphism, is based on 3 factors: (1) absence of the change in at least 50 independent control chromosomes; (2) presence of the amino acid in the bacterial enzyme, attesting to its evolutionary significance and (3) change in amino acid not conservative. Although expression of the substitutions is required to formally prove that they are not benign, the criteria above allow us to postulate that the changes described in this report are likely to affect activity.

### BRIEF DESCRIPTION OF THE DRAWINGS

FIGS. 1A to 1F illustrate the first cDNA coding sequence (SEQ ID NO:1 and NO:2) for methylenetetrahydrofolate reductase (MTHFR);

FIG. 2 is the alignment of amino acids for human methylenetetrahydrofolate reductase (MTHFR), the metF genes from E. Coli (ECOMETF), and S. Typhimurium (STYMETF), and an unidentified open reading frame in Saccharomyces cerevisiae that is divergently transcribed from an excision repair gene (ysRAD1);

FIGS. 3A and 3B illustrate the sequencing and restriction enzyme analysis for the Arg to Ter substitution;

FIGS. 4A and 4B illustrate the sequencing and restriction enzyme analysis for the Arg to Gln substitution;

FIGS. 5A and 5B illustrate the sequence change and restriction enzyme analysis for the alanine to valine substitution;

FIGS. 6A to 6C illustrate the total available sequence (SEQ ID NO:3 and NO:4) of human MTHFR cDNA;

FIGS. 7A and 7B illustrate the expression analysis of MTHFR cDNA in E. Coli, respectively (7A) the Western blot of bacterial extracts and tissues, and (7B) the thermolability assay of bacterial extracts;

FIGS. 8A to 8D illustrate the identification of a 5′ splice site mutation leading to a 57-bp in-frame deletion of the CDNA;

FIGS. 9A to 9D illustrate the diagnostic restriction endonuclease analysis of 4 mutations;

FIGS. 10A to 10D illustrate the ASO hybridization analysis of 2 mutations;

FIG. 11 illustrates the region of homology between human methylenetetrahydrofolate reductase (MTHFR) and human dihydrofolate reductase (DHFR);

FIGS. 12A-12B illustrate the exonic sequences of the human MTHFR gene with their flanking intronic sequences;

FIGS. 13A-13B illustrate the exonic sequences of the mouse MTHFR gene with their flanking intronic sequences;

FIG. 14 illustrates intron sizes and locations for both human and mouse genes; and

FIG. 15 illustrates the alignment of MTHFR amino acid sequences for the human MTHFR (hMTHFR), mouse MTHFR (mMTHFR) and the MetF gene of bacteria (bMTHFR).

FIG. 16 is a graph that shows the percent survival of SW620 colon carcinoma cells following three rounds of treatment for five hours each round with varying concentrations of an antisense phosphorothioate oligonucleotide to exon 5 of MTHFR. The cells were allowed to recover after the final treatment for a period of two days before the cells were counted. The values are expressed as the percent of cells surviving compared to the number of cells which survived after treatment with a control oligonucleotide (phosphorothioate 5′-GTGACGTAGGACAGCGATGG-3′).

FIG. 17 is a graph showing the percent survival of LOVO colon carcinoma cells treated with an MTHFR antisense oligonucleotide after a recovery period of three days, as described for FIG. 16.

FIG. 18 is a graph showing the percent survival of BEC 2 neuroblastoma cells treated with varying concentrations of an MTHFR antisense oligonucleotide and allowed to recover for two days, as described for FIG. 16.

FIG. 19 is a graph that shows the percent survival of SK-N-F1 neuroblastoma cells treated with varying concentrations of an MTHFR antisense oligonucleotide after a recovery period of four days, as described for FIG. 16.

FIG. 20 is a graph showing the percent survival of MCF7 breast cancer cells that were treated with different concentrations of an MTHFR antisense oligonucleotide and allowed to recover for 2.5 days, as described for FIG. 16.

FIG. 21 is a graph showing the percent survival of SKBr3 breast cancer cells treated with varying concentrations of an MTHFR antisense oligonucleotide after a recovery period of 2.5 days, as described for FIG. 16.

FIG. 22 is a graph showing the percent survival of U87-lacZ glioma cells treated with varying concentrations of an MTHFR antisense oligonucleotide after a recovery period of two days, as described for FIG. 16. For this experiment an oligonucleotide with six base pair mismatches (phosphorothioate 5′-TGCTGTCGGAGCGATAGGTC-3′) was used as the control oligonucleotide.

FIG. 23 is a graph showing the percent survival of WG1554 fibroblast cells homozygous for a nonsense mutation in MTHFR which were treated with 400 nM MTHFR antisense or control oligonucleotide and allowed to recover for three days, as described for FIG. 16.

FIG. 24A is an illustration of a Western blot showing that the level of MTHFR protein is reduced in cells transfected with a specific MTHFR antisense oligonucleotide against exon 5 (lane 3) compared to mock-transfected cells (lane 1) or cells transfected with a control mismatched oligonucleotide (lane 2). FIG. 24B is an illustration of a Western blot performed on the same extracts using an antibody reactive with the unrelated protein actin as an internal control. This Western blot indicates that equal amounts of cell extract were loaded in the wells and that the inhibition was specific for MTHFR, rather than inhibition of general protein synthesis.

FIG. 25 is a graph showing the percent survival of SW620 colon carcinoma cells treated with varying concentrations of an antisense oligonucleotide to the valine (677T) allele of MTHFR, an antisense oligonucleotide to the alanine (677C) allele, or the corresponding control oligonucleotide and allowed to recover for three days. This experiment was performed as described for FIG. 16 using an MTHFR antisense oligonucleotide to exon 5.

FIG. 26A is an illustration of a Western blot showing that the level of MTHFR protein in SW620 colon carcinoma cells, which are homozygous for a valine MTHFR allele, is decreased by administration of an antisense oligonucleotide (ASO) targeting the valine allele but not the administration of an antisense oligonucleotide targeting the alanine allele or a control oligonucleotide. As illustrated in FIG. 26B, similar levels of cell extract were loaded in each lane of the Western blot.

### DETAILED DESCRIPTION OF THE INVENTION

Sequencing of peptides from porcine MTHFR

Homogeneous native porcine MTHFR was digested with trypsin to generate a 40 kDa N-terminal fragment and a 31 kDa C-terminal fragment; the 31 kDa fragment is a proteolytic product of the 37 kDa fragment. The fragments were separated by SDS-PAGE, electroeluted, and the denatured fragments were digested with lysyl endopeptidase (LysC). The resulting peptides were separated by reversed-phase HPLC and subjected to sequence analysis by Edman degradation (details contained in Goyette P et al., Nature Genetics, 1994, 7:195-200).

Isolation and sequencing of cDNAs

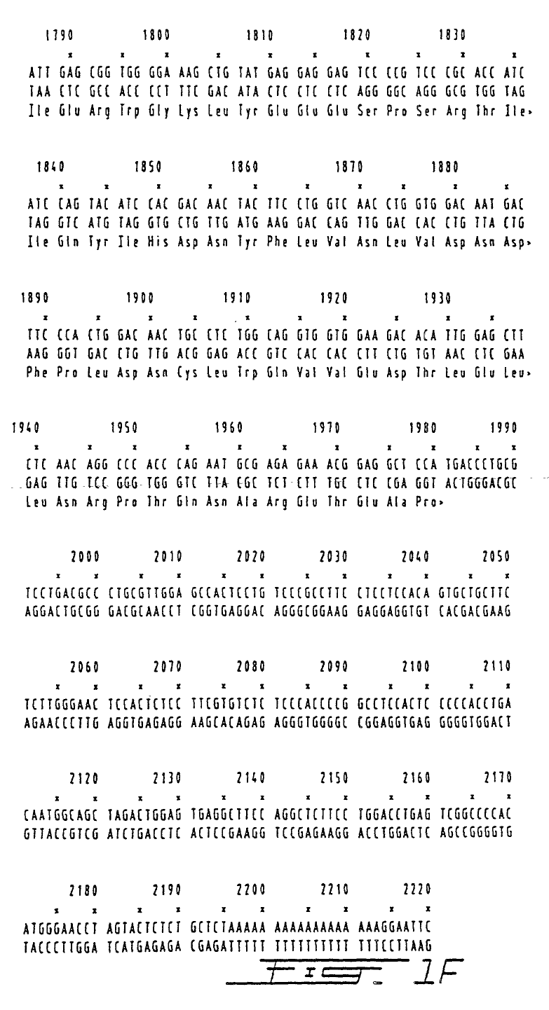

Two degenerate oligonucleotides were synthesized based on the sequence of a 30 amino acid porcine MTHFR peptide (first underlined peptide in FIG. 2). These were used to generate a 90 bp PCR product, encoding the predicted peptide, from reverse transcription-PCR reactions of 500 ng pig liver polyA+ RNA. A pig-specific (non-degenerate, antisense) PCR primer was then synthesized from this short cDNA sequence. Using this primer and a primer for phage arms, a human liver λgt10 cDNA library (Clontech) was screened by PCR; this technique involved the generation of phage lysate stocks (50,000 pfu) which were boiled for 5 min and then used directly in PCR reactions with these two primers. PCR fragments were then sequenced directly (Cycle Sequencing™ kit, GIBCO), and a positive clone was identified by comparison of the deduced amino acid sequence to the sequence of the pig peptide (allowing for inter-species variations). The positive stock was then replated at lower density and screened with the radiolabelled positive PCR product by plaque hybridization until a well-isolated plaque was identified. Phage DNA was purified and the insert was then subcloned into pBS+ (Bluescript) and sequenced on both strands (Cycle SequencingTm kit, GIBCO and Sequenase T, Pharmacia). The deduced amino acid sequence of the human cDNA was aligned to the porcine peptide sequences, the metf genes from E. coli (ecometf, accession number V01502) and S. Typhimurium (stymetF, accession number X07689) and with a previously unidentified open reading frame in Saccharomyces cerevisiae that is divergently transcribed with respect to the excision repair gene, ysRAD1 (accession number K02070). The initial alignments were performed using BestFit™ in the GCG computer package, and these alignments were adjusted manually to maximize homologies.

In summary, degenerate oligonucleotide primers were designed to amplify a sequence corresponding to a 30-amino acid segment of a porcine peptide from the N-terminal region of the enzyme (first porcine peptide in FIG. 2). A 90-bp porcine cDNA fragment was obtained from reverse transcription/PCR of pig liver RNA. Sequencing of the PCR fragment confirmed its identity by comparison of the deduced amino acid sequence to the porcine peptide sequence. A nondegenerate oligonucleotide primer, based on the internal sequence of the porcine cDNA, was used in conjunction with primers for the phage arms to screen a human liver λgt10 CDNA library by PCR. The insert of the positive clone was isolated and sequenced. The sequence consisted of 1266 bp with one continuous open reading frame.

Homology with MTHFR in other species

The deduced amino acid sequence of the human cDNA was aligned with the metF genes from E. coli and S. typhimurium, as well as with a previously unidentified ORF in Saccharomyces cerevisiae that is divergently transcribed with respect to the excision repair gene, ysRAD1 (FIG. 2). The sequences homologous to 5 porcine peptides are underlined in FIG. 2. Three segments (residues 61-94, 219-240, and 337-351) correspond to internal peptide sequence from the N-terminal 40-kDa domain of the porcine liver enzyme. Residues 374-393 correspond to the upstream portion of the LysC peptide from the C-terminal domain of the porcine liver enzyme that is labeled when the enzyme is irradiated with UV light in the presence of (³H-methyl)AdoMet; as predicted from the AdoMet labeling studies, this peptide lies at one end (N-terminal) of the 37 kDa domain. A fifth region of homology (residues 359-372) was also identified, but the localization of the porcine peptide within the native protein had not been previously determined.

Methylenetetrahydrofolate reductase (MTHFR) is an enzyme involved in amino acid metabolism, that is critical for maintaining an adequate methionine pool, as well as for ensuring that the homocysteine concentration does not reach toxic levels. The high degree of sequence conservation, from E. coli to Homo sapiens, attests to the significance of MTHFR in these species. The enzyme in E. coli (encoded by the metf locus) is a 33-kDa peptide that binds reduced FAD and catalyzes the reduction of methylenetetrahydrofolate to methyltetrahydrofolate. The metf enzyme differs from the mammalian enzyme in that NADPH or NADH cannot reduce it, and its activity is not allosterically regulated by S-adenosylmethionine. The native porcine enzyme is susceptible to tryptic cleavage between the N-terminal 40 kDa domain and the C-terminal 37 kDa domain, and this cleavage results in the loss of allosteric regulation by adenosylmethionine, but does not result in loss of catalytic activity. Since the homology between the bacterial and mammalian enzymes is within the N-terminal domain, this region must contain the flavin binding site and residues necessary to bind the folate substrate and catalyze its reduction. The domain structure of the human enzyme has not been elucidated, although the human enzyme has been reported to have a molecular mass of 150 kDa and is likely to be a homodimer of 77 kDa.

The predicted point of cleavage between the two domains lies between residues 351 and 374 of the human sequence, based on the localization of peptides obtained from the isolated domains of the porcine enzyme. This region, containing the highly charged sequence KRREED, is predicted to have the highest hydrophilicity and surface probability of any region in the deduced human sequence.

The N-terminus of the porcine protein has been sequenced, and the region encoding this part of the protein is missing from the human cDNA. It is estimated that this cDNA is missing only a few residues at the N-terminus, since the predicted molecular mass of the deduced sequence upstream of the putative cleavage site (KRREED) is 40 kDa, and the measured molecular mass of the porcine N-terminal domain is also 40 kDa. When the bacterial, yeast and human sequences are aligned, the deduced human sequence contains an N-terminal extension of 40 amino acids; it is suspected that this extension contains determinants for NADPH binding. Many pyridine nucleotide-dependent oxidoreductases contain such determinants at the N-terminus of the protein.

The C-terminus of the human sequence contains a peptide that is labeled when the protein is irradiated with ultraviolet light in the presence of tritiated AdoMet. The cDNA sequence reported here contains only about 7 kDa of the predicted 37-kDa mass of this domain, indicating that this cDNA is truncated at the 3′ terminus as well. A number of peptides from the C-terminal porcine domain have also not been detected. As might be expected, given that the prokaryotic enzymes do not appear to be allosterically regulated by AdoMet, there are no significant homologies between the C-terminal region in this cDNA and the prokaryotic metF sequences. The alignment shown in FIG. 2 shows that the homologous sequences terminate just prior to the putative cleavage site of the human enzyme.

Chromosomal assignment

In situ hybridization to metaphase human chromosomes was used for localization of the human gene. The analysis of the distribution of 200 silver grains revealed a significant clustering of grain 40 grains, in the p36.3-36.2 region of chromosome 1 (p<0.0001), with the majority of grains, 25 grains, observed over 1p36.3.

The isolation of the human cDNA has allowed us to localize the gene to chromosome 1p36.3. The observation of one strong signal on that chromosome with little background is highly suggestive of a single locus with no pseudogenes. Southern blotting of human DNA revealed fragments of approximately 10 kb, predicting a gene of average size, since this cDNA encodes approximately half of the coding sequence.

Additional CDNA sequences and constructs for expression analysis

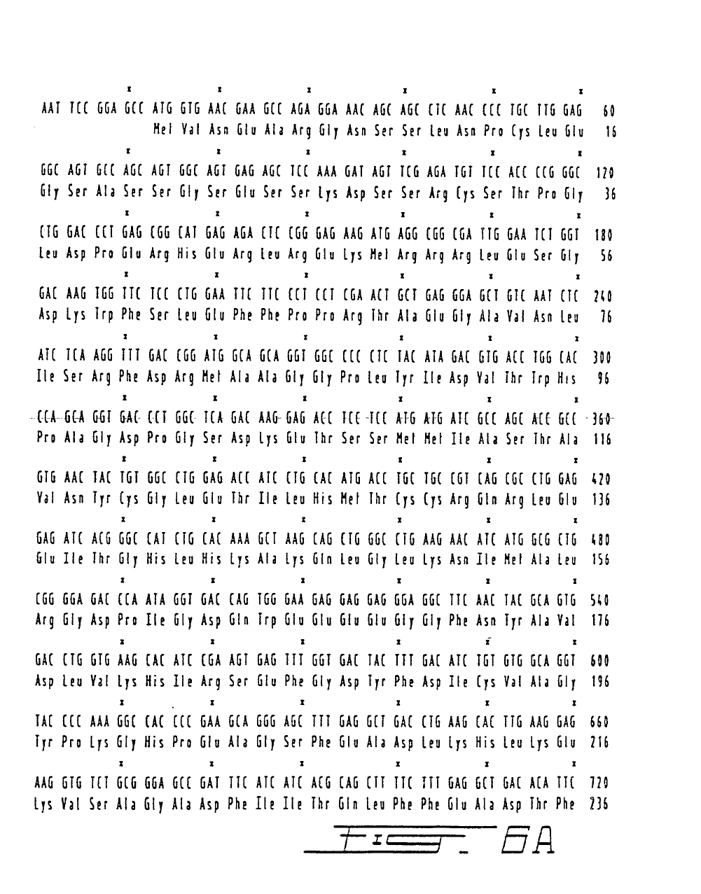

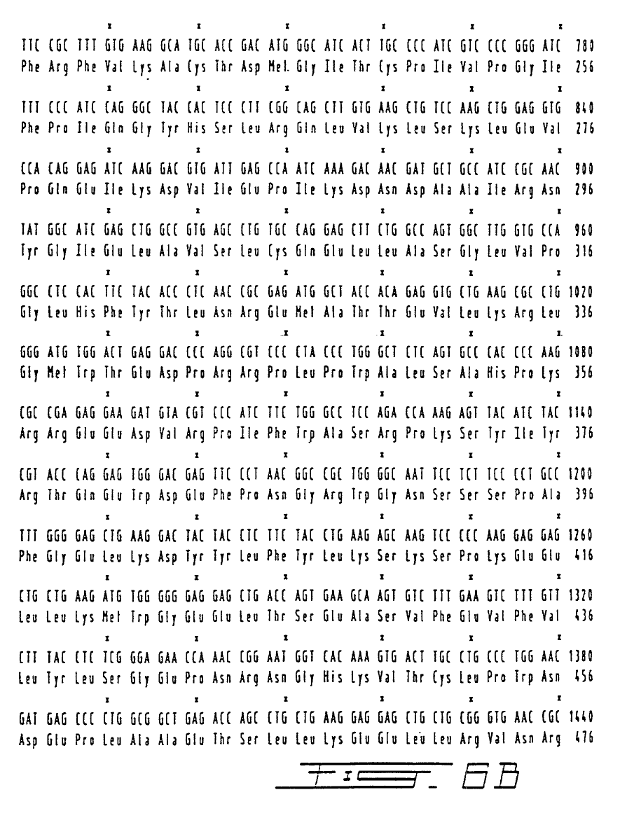

A human colon carcinoma cDNA library (gift of Dr. Nicole Beauchemin, McGill University) was screened by plaque hybridization with the original 1.3-kb cDNA to obtain additional coding sequences. A cDNA of 2.2 kb was isolated, which contained 1.3 kb of overlapping sequence to the original cDNA and 900 additional bp at the 3′ end (FIG. 6). The amino acid sequence is identical to that of the original cDNA for the overlapping segment (codons 1-415) except for codon 177 (ASP) which was a GLY codon in the original cDNA. Analysis of 50 control chromosomes revealed an ASP codon at this position. The cDNA has an open reading frame of 1980 bp, 100 bp of 3′ UTR and a poly A tail.

Sequencing was performed on both strands for the entire cDNA. Additional 5′ sequences (800 bp) were obtained from a human kidney cDNA library (Clontech) but these sequences did not contain additional coding sequences and were therefore used for the PCR-based mutagenesis only (as described below) and not for the expression analysis. The two cDNAs (2.2 kb and 800 bp) were ligated using the EcoRI site at bp 199 and inserted into the Bluescript™ vector (Stratagene). The 2.2 kb cDNA was subcloned into the expression vector pTrc99A (Pharmacia) using the NcoI site at bp 11 and the XbaI site in the polylinker region of both the Bluescript™ and the pTrc99A vectors. Sequencing was performed across the cloning sites to verify the wild-type construct.

### UTILITY OF INVENTION IN IDENTIFICATION OF MUTATIONS

I. Identification of first two mutations in severe MTHFR deficiency

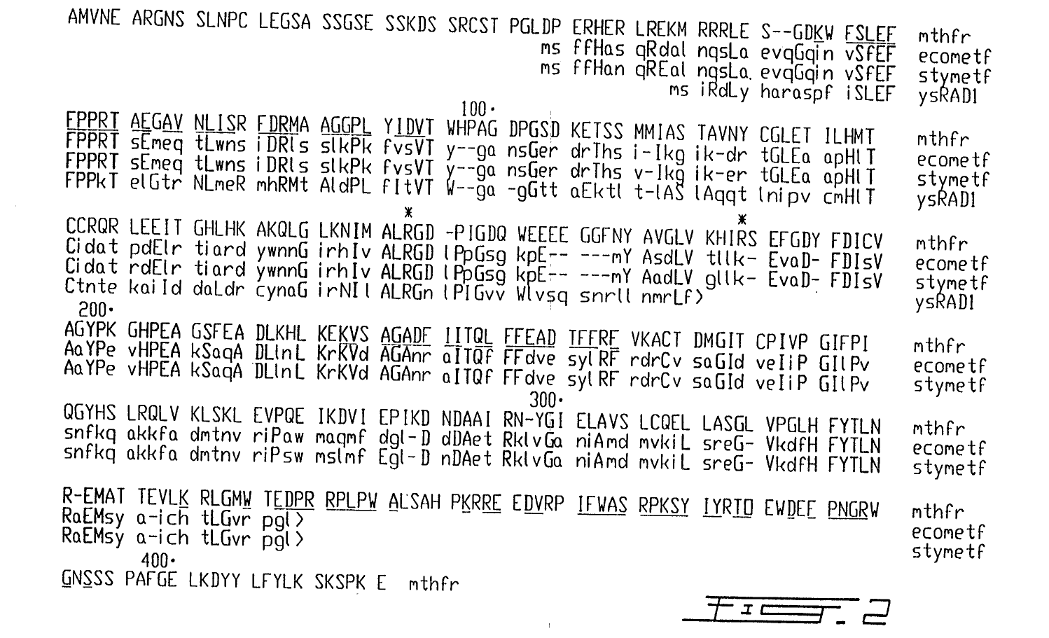

Total RNA of skin fibroblasts from MTHFR-deficient patients was reverse-transcribed and amplified by PCR for analysis by the single strand conformation polymorphism (SSCP) method (Orita, M. et al., Genomics, 1989, 5:8874-8879). Primers were designed to generate fragments of 250-300 bp and to cover the available cDNA sequences with small regions of overlap for each fragment at both ends. The first mutation identified by SSCP was a C to T substitution at bp 559 in patient 1554; this substitution converted an arginine codon to a termination codon (FIG. 3A). Since the mutation abolished a Foki site, restriction digestion was used for confirmation of the change and for screening additional patients for this mutation; a second patient (1627) was identified in this manner (FIG. 3B). The SSCP pattern for patient 1554 and the restriction digestion pattern for both patients was consistent with a homozygous mutant state or with a genetic compound consisting of the nonsense mutation with a second mutation that did not produce any detectable RNA (null allele). Studies in the parents are required for confirmation.

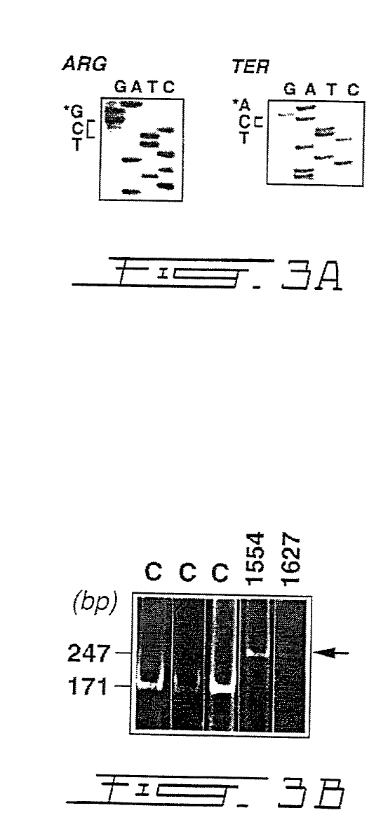

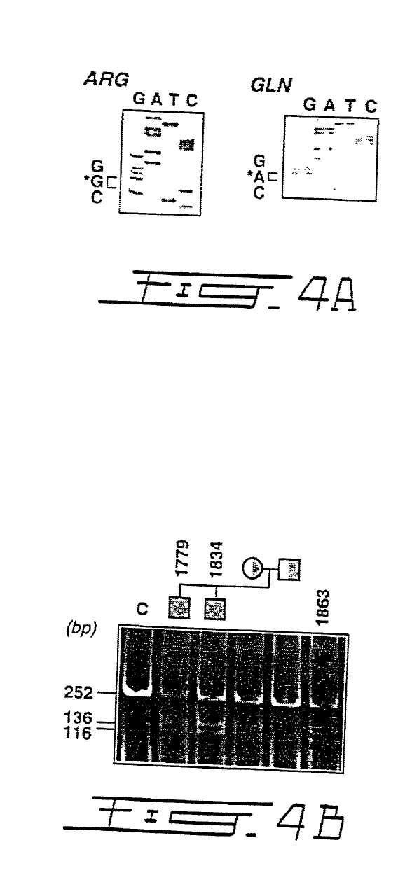

The second substitution (FIG. 4A) was a G to A transition at bp 482 in patient 1834 that converted an arginine into a glutamine residue. The substitution created a PstI site that was used to verify the substitution and to identify a second patient (1863) with this change (FIG. 4B). The SSCP analysis and the restriction digestion pattern were consistent with a heterozygous state for both patients. The arginine codon affected by this change is an evolutionarily conserved residue, as shown in FIG. 2. This observation, in conjunction with the fact that the codon change is not conservative, makes a strong argument that the substitution is a pathologic change rather than a benign polymorphism. Furthermore, 35 controls (of similar ethnic background to that of the probands) were tested for this substitution by Southern blotting of PstI-digested DNA; all were negative.

The family of patient 1834 was studied. The symptomatic brother and the mother of the proband were all shown to carry this substitution, whereas the father was negative for the change (FIG. 4B). In the family of 1863, the mother of the proband was shown to be a carrier, while the father and an unaffected brother were negative.

Cell lines

Cell line 1554 is from a Hopi male who was admitted at age three months with homocystinuria, seizures, dehydration, corneal clouding, hypotonia and Candida sepsis. Folate distribution in cultured fibroblasts showed a Pediococcus cerivisiae/Lactobacillus casei (PC/LC) ratio of 0.52 (Control 0.14). There was no measurable methylenetetrahydrofolate reductase (MTHFR) activity (Control values=9.7 and 15.1 nmoles/h/mg protein; residual activity after treatment of control extracts at 55° C. for 20 min.=28% and 31%).

Cell line 1627 is from a Choctaw male who presented with poor feeding, apnea, failure to thrive, dehydration and homocystinuria at five weeks of age. He was subsequently found to have superior sagittal sinus thrombosis and hydrocephalus. The PC/LC ratio was 0.61 and the specific activity of MTHFR was 0.1 nmoles/h/mg protein. There is consanguinity in that the maternal and paternal grandmothers are thought to be “distantly related”.

Cell line 1779 is from a French Canadian male with homocystinuria who first had limb weakness, uncoordination, paresthesiae, and memory lapses at age 15 years, and was wheelchair-bound in his early twenties. His brother (cell line 1834) also has homocystinuria, but is 37 years old and asymptomatic. Specific activity of MTHFR was 0.7 and 0.9 nmole/h/mg protein for 1779 and 1834, respectively; the residual activity after heat treatment at 55° C. was 0.9% and 0% for 1779 and 1834, respectively.

Cell line 1863 is from a white male who was diagnosed at age 21 years because of a progressive gait disturbance, spasticity, cerebral white matter degeneration, and homocystinuria. He had a brother who died at age 21 years of neurodegenerative disease. Specific activity of MTHFR in fibroblast extracts was 1.76 nmoles/h/mg protein and the residual enzyme activity after treatment at 55° C. was 3.6%.

Mutation analysis

Primers were designed from the cDNA sequence to generate 250-300 bp fragments that overlapped 50-75 bp at each end. The primer pairs were used in reverse transcription-PCR of 5 μg patient total fibroblast RNA. The PCR products were analyzed by a non-isotopic rapid SSCP protocol (PhastSystem™, Pharmacia), which uses direct silver staining for detection of single strands. Any PCR products from patients showing a shift on SSCP gels were purified by NuSieve (FMC Bioproducts) and sequenced directly (Cycle Sequencing™ kit, GIBCO) to identify the change. If the change affected a restriction site, then a PCR product was digested with the appropriate restriction endonuclease and analyzed on polyacrylamide gels. To screen for the Arg to Gln mutation in controls, 5 μg of PstI-digested DNA was run on 0.8% agarose gels and analyzed by Southern blotting using the radiolabelled cDNA by standard techniques.

II. Seven additional mutations at the methylenetetrahydrofolate reductase (MTHFR) locus with genotype: phenotype correlation in severe MTHFR deficiency

It is reported hereinbelow the characterization of 7 additional mutations at this locus: 6 missense mutations and a 5′ splice site defect which activates a cryptic splice site in the coding sequence. A preliminary analysis of the relationship between genotype and phenotype for all 9 mutations identified thus far at this locus is also reported. A nonsense mutation and 2 missense mutations (proline to leucine and threonine to methionine) in the homozygous state are associated with extremely low activity (0-3%) and onset of symptoms within the first year. Other missense mutations (arginine to cysteine and arginine to glutamine) are associated with higher enzyme activity and later onset of symptoms.

7 additional mutations at the MTHFR locus are described and the association between genotype, enzyme activity, and clinical phenotype in severe MTHFR deficiency is examined.

Patient description

The clinical and laboratory findings of the patients have been reported in the published literature. Residual MTHFR activity was previously measured in cultured fibroblasts at confluence.

Patient 354, an African-American girl, was diagnosed at age 13 years with mild mental retardation. Her sister, patient 355 was diagnosed at age 15 years with anorexia, tremor, hallucinations and progressive withdrawal. In patient 354, residual MTHFR activity was 19% and in her sister, 355, it was 14% of control values. The residual activity after heating had equivalent thermal stability to control enzyme.

Patient 1807, a Japanese girl whose parents are first cousins, had delayed walking and speech until age 2 years, seizures at age 6 years and a gait disturbance with peripheral neuropathy at age 16 years. Residual activity of MTHFR was 3% and the enzyme was thermolabile.

Patient 735, an African-Indian girl, was diagnosed at age 7 months with microcephaly, progressive deterioration of mental development, apnea and coma. Residual activity of MTHFR was 2% of control levels. Thermal properties were not determined.

Patient 1084, a Caucasian male, was diagnosed at age 3 months with an infantile fibrosarcoma. He was found to be hypotonic and became apneic. He died at the age of 4 months. Residual activity of MTHFR was not detectable. Thermal properties were not determined.

Patient 356, the first patient reported with MTHFR deficiency, is an Italian-American male who presented at age 16 years with muscle weakness, abnormal gait and flinging movements of the upper extremities. MTHFR residual activity was 20% of control values; activity was rapidly and exponentially inactivated at 550.

Patient 458, a Caucasian male, was diagnosed at age 12 years with ataxia and marginal school performance. Residual MTHFR activity was approximately 10%, and the activity was thermolabile.

Patient 1396, a Caucasian female, was described as clumsy and as having a global learning disorder in childhood. At age 14 years, she developed ataxia, foot drop, and inability to walk. She developed deep vein thrombosis and bilateral pulmonary emboli. Residual activity of MTHFR was 14% and the enzyme was thermolabile.

Genomic structure and intronic primers

Exon nomenclature is based on available cDNA sequence in Goyette et al. (Nature Genetics, 1994, 7:195-200). Exon 1 has been arbitrarily designated as the region of cDNA from bp 1 to the first intron. Identification of introns was performed by amplification of genomic DNA using cDNA primer sequences. PCR products that were greater in size than expected cDNA sizes were sequenced directly.

Mutation detection

Specific exons (see Table 1 for primer sequences) were amplified by PCR from genomic DNA and analyzed by the SSCP protocol. SSCP was performed with the Phastgel™ system (Pharmacia), a non-isotopic rapid SSCP protocol, as previously described (Goyette P et al., Nature Genetics, 1994, 7:195-200), or with ³⁵S-labeled PCR products run on 6% acrylamide: 10% glycerol gels at room temperature (6 watts, overnight). In some cases, the use of restriction endonucleases, to cleave the PCR product before SSCP analysis, enhanced the detection of band shifts. PCR fragments with altered mobility were sequenced directly (GIBCO, Cycle Sequencing™ kit). If the sequence change affected a restriction endonuclease site, then the PCR product was digested with the appropriate enzyme and analyzed by PAGE. Otherwise, allele-specific oligonucleotide (ASO) hybridization was performed on a dot blot of the PCR-amplified exon.

TABLE 1
PCR Primers for DNA amplification and mutation analysis
of MTHFR

| | | | | Frag- |
|---|---|---|---|---|
| | | | | ment |
| | Primer | | Loca- | Size |
| Exon | Type | Primer Sequence (5′→3′) | tion | (bp) |
| 1 | Sense | AGCCTCAACCCCTGCTTGGAGG | C | 271 |
| | Antisense | (SEQ ID NO:5) | I | |
| | | TGACAGTTTGCTCCCCAGGCAC | | |
| | | (SEQ ID NO:6) | | |
| 4 | Sense | TGAAGGAGAAGGTGTCTGCGGGA | C | 198 |
| | Antisense | (SEQ ID NO:7) | I | |
| | | AGGACGGTGCGGTGAGAGTGG | | |
| | | (SEQ ID NO:8) | | |
| 5 | Sense | CACTGTGGTTGGCATGGATGATG | I | 392 |
| | Antisense | (SEQ ID NO:9) | I | |
| | | GGCTGCTCTTGGACCCTCCTC | | |
| | | (SEQ ID NO:10) | | |
| 6 | Sense | TGCTTCCGGCTCCCTCTAGCC | I | 251 |
| | Antisense | (SEQ ID NO:11) | I | |
| | | CCTCCCGCTCCCAAGAACAAAG | | |
| | | (SEQ ID NO:12) | | |

TABLE 2
Summary of genotypes, enzyme activity, age at onset, and
background of patients with MTHFR deficiency

| | | Amino | | | | |
|---|---|---|---|---|---|---|
| Pa- | | acid | % Ac- | Age at | | |
| tient^{a} | BPChanges^{b} | changes | tivity | Onset | | Background |
| 1807 | C764T/ | Pro→Leu/ | 3 | within | | Japanese |
| | C764T | Pro→Leu | | 1st year | | |
| 735 | C692T/ | Thr→Met/ | 2 | 7 | months | African |
| | C692T | Thr→Met | | | | Indian |
| 1084 | C692T/ | Thr→Met/ | 0 | 3 | months | Caucasian |
| | C692T | Thr→Met | | | | |
| 1554 | C559T/ | Arg→Ter/ | 0 | 1 | month | Native |
| | C559T | Arg→Ter | | | | American |
| | | | | | | (Hopi) |
| 1627 | C559T/ | Arg→Ter/ | 1 | 1 | month | Native |
| | C559T | Arg→Ter | | | | American |
| | | | | | | (Choctaw) |
| 356 | C985T/ | Arg→Cys/ | 20 | 16 | yrs | Italian |
| | C985T | Arg→Cys | | | | American |
| 458 | C1015T/ | Arg→Cys/ | 10 | 11 | yrs | Caucasian |
| | G167A | Arg→Gln | | | | |
| 1396 | C1081T/ | Arg→Cys/ | 14 | 14 | yrs | Caucasian |
| | G167A | Arg→Gln | | | | |
| 1779^{c} | G482A/? | Arg→Gln/ | 6 | 15 | yrs | French |
| | | ? | | | | Canadian |
| 1834^{c} | G482A/? | Arg→Gln/ | 7 | Asympto- | | French |
| | | ? | | matic | | Canadian |
| | | | | at 37 yrs | | |
| 1863 | G482A/? | Arg→Gln/ | 14 | 21 | yrs | Caucasian |
| | | ? | | | | |
| 354^{d} | 792 + | 5′ splice | 19 | 13 | yrs | African |
| | 1G→A/? | site/? | | | | American |
| 355^{d} | 792 + | 5′ splice | 14 | 11 | yrs | African |
| | 1G→A/? | site/? | | | | American |

^{a}Patients 1554, 1627, 1779, 1834 and 1863 were previously reported by Goyette et al. (1994).
^{b}? = unidentified mutation.
^{c}Patients 1779 and 1834 are sibs.
^{d}Patients 354 and 355 are sibs.

(1) 5′ splice site mutation

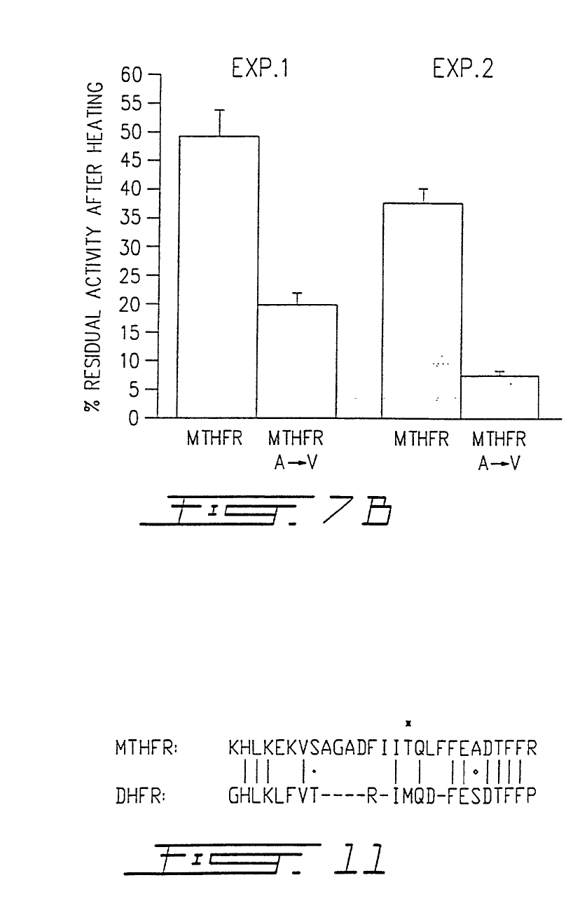

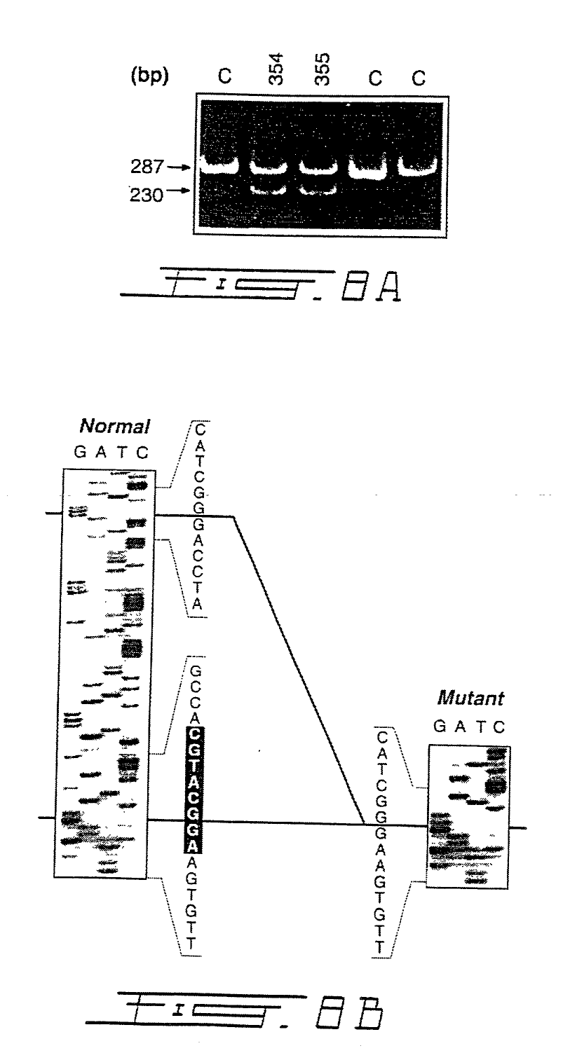

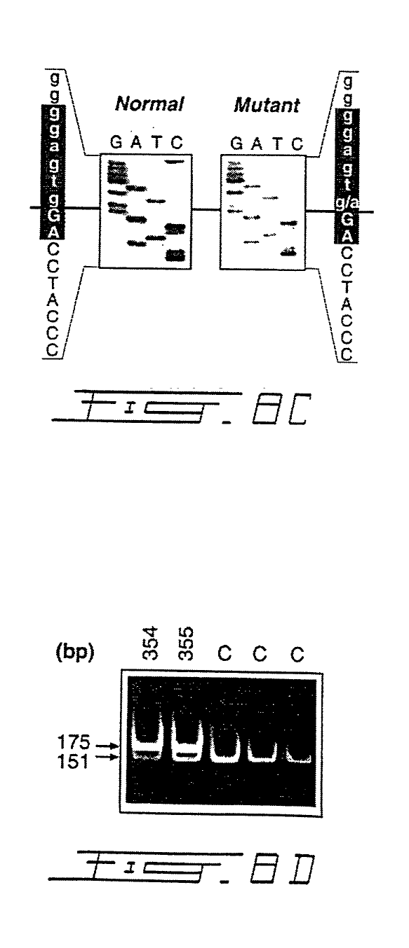

Amplification of cDNA, bp 653-939, from reverse-transcribed total fibroblast RNA revealed 2 bands in sisters 354 and 355: a smaller PCR fragment (230 bp) in addition to the normal 287 bp allele (FIG. 8A). FIG. 8A is the PAGE analysis of amplification products of cDNA bp 653-939, from reverse transcribed RNA. Controls have the expected 287-bp fragment while patients 354 and 355 have an additional 230-bp fragment. Sequencing of the smaller fragment identified a 57-bp in-frame deletion which would remove 19 amino acids (FIG. 8B). FIG. 8B is the direct sequencing of the PCR products from patient 354. The 57-bp deletion spans bp 736-792 of the cDNA. An almost perfect 5′ splice site (boxed) is seen at the 5′ deletion breakpoint. Analysis of the sequence at the 5′ deletion breakpoint in the undeleted fragment revealed an almost perfect 5′ splice site consensus sequence (AG/gcatgc). This observation suggested the presence of a splicing mutation in the natural 5′ splice site that might activate this cryptic site, to generate the deleted allele. The sequence following the deletion breakpoint, in the mutant allele, corresponded exactly to the sequence of the next exon. Amplification of genomic DNA, using the same amplification primers as those used for reverse-transcribed RNA, generated a 1.2-kb PCR product indicating the presence of an intron. Direct sequencing of this PCR fragment in patient 354 identified a heterozygous G→A substitution in the conserved GT dinucleotide of the intron at the 5′ splice site (FIG. 8C). FIG. 8C is the sequencing of the 5′ splice site in control and patient 354. The patient carries a heterozygous G→A substitution in the 5′ splice site (boxed). Intronic sequences are in lower case. This substitution abolished a HphI restriction endonuclease site which was used to confirm the mutation in the 2 sisters (FIG. 8D). FIG. 8D is the HphI restriction endonuclease analysis on PCR products of DNA for exon 4 of patients 354 and 355, and of 3 controls (C). The 198-bp PCR product has 2 HphI sites. The products of digestion for the control allele are 151, 24 and 23 bp. The products of digestion for the mutant allele are 175 and 23 bp due to the loss of a HphI site. The fragments of 24 and 23 bp have been run off the gel.

(2) Patients with homozygous coding substitutions

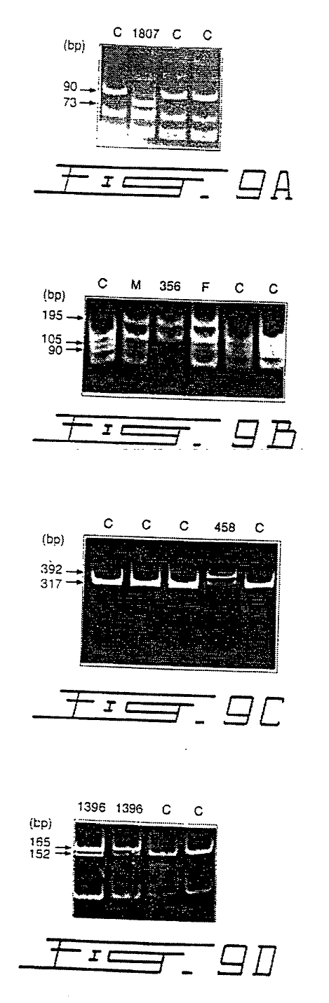

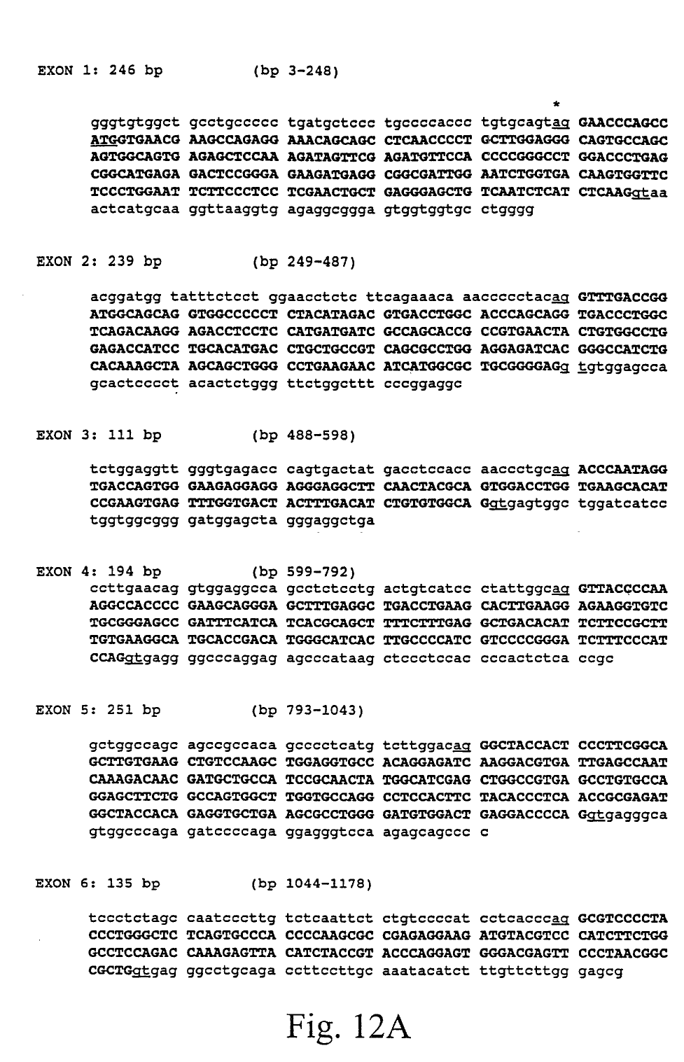

SSCP analysis of exon 4 for patient 1807 revealed an abnormally-migrating fragment, which was directly sequenced to reveal a homozygous C→T substitution (bp 764) converting a proline to a leucine residue. This change creates a MnlI restriction endonuclease site, which was used to confirm the homozygous state of the mutation (FIG. 9A). FIG. 9A is the MnlI restriction analysis of exon 4 PCR products for patient 1807 and 3 controls (C). Expected fragments: control allele, 90, 46, 44, 18 bp; mutant allele, 73, 46, 44, 18, 17 bp. An additional band at the bottom of the gel is the primer. Fifty independent control Caucasian chromosomes and 12 control Japanese chromosomes were tested by restriction analysis; all were negative for this mutation. Homozygosity in this patient is probably due to the consanguinity of the parents.

Patients 735 and 1084 had the same mutation in exon 4, in a homozygous state: a C→T substitution (bp 692) which converted an evolutionarily conserved threonine residue to a methionine residue, and abolished a NlaIII restriction endonuclease site. Allele-specific oligonucleotide hybridization to amplified exon 4 (FIGS. 10A and 10B) was used to confirm the mutation in these 2 patients and to screen 60 independent chromosomes, all of which turned out to be negative. FIG. 10A is the hybridization of mutant oligonucleotide (692T) to exon 4 PCR products from patients 735, 1084 and 30 controls. Only DNA from patients 735 and 1084 hybridized to this probe. FIG. 10B is the hybridization of normal oligonucleotide (692C) to stripped dot blot from A. All control DNAs hybridized to this probe.

Patient 356 showed a shift on SSCP analysis of exon 5. Direct sequencing revealed a homozygous C→T substitution (bp 985) which converted an evolutionarily conserved arginine residue to cysteine; the substitution abolished an AciI restriction endonuclease site. This was used to confirm the homozygous state of the mutation in patient 356 (FIG. 9B) and its presence in the heterozygous state in both parents. Fifty independent control chromosomes, tested in the same manner, were negative for this mutation. FIG. 9B is the AciI restriction analysis of exon 5 PCR products for patient 356, his father (F), his mother (M), and 3 controls (C). Expected fragments: control allele, 129, 105, 90, 68 bp; mutant allele, 195, 129, 68 bp.

(3) Patients who are genetic compounds

Patient 458 is a compound heterozygote of a mutation in exon 5 and a mutation in exon 1. The exon 5 substitution (C→T at bp 1015) resulted in the substitution of a cysteine residue for an arginine residue; this abolished a HhaI restriction endonuclease site, which was used to confirm the mutation in patient 458 (FIG. 9C) and to show that 50 control chromosomes were negative. FIG. 9C is the HhaI restriction analysis of exon 5 PCR products for patient 458 and 4 controls (C). Expected fragments: control allele, 317 and 75 bp; mutant allele 392 bp. The 75-bp fragment is not shown in FIG. 9C. The second mutation was a heterozygous G→A substitution (bp 167) converting an arginine to a glutamine residue. Allele-specific oligonucleotide hybridization to amplified exon 1 confirmed the heterozygous state of this mutation in patient 458 and identified a second patient (1396) carrying this mutation also in the heterozygous state (FIGS. 10C and 10D). FIG. 10C is the hybridization of mutant oligonucleotide (167A) to exon 1 PCR products from patients 458, 1396 and 31 controls. FIG. 10D is the hybridization of normal oligonucleotide (167G) to stripped dot blot from C. None of the 62 control chromosomes carried this mutation.

The second mutation in patient 1396 was identified in exon 6: a heterozygous C→T substitution (bp 1081) that converted an arginine residue to a cysteine residue, and abolished a HhaI restriction endonuclease site. Restriction analysis confirmed the heterozygous substitution in 1396 (FIG. 9D) and showed that 50 control chromosomes were negative. Tooth decay does not occur in patients having a saliva above pH 5.0. FIG. 9D is the HhaI restriction analysis of exon 6 PCR products for patient 1396 and 2 controls (C). Expected fragments: control allele, 152, 86, 13 bp; mutant allele 165, 86 bp. The 13-bp fragment has been run off the gel.

(4) Additional sequence changes

HhaI analysis of exon 6, mentioned above, revealed a DNA polymorphism. This change is a T→C substitution at bp 1068 which does not alter the amino acid (serine), but creates a HhaI recognition site. T at bp 1068 was found in 9% of tested chromosomes. Sequence analysis identified 2 discrepancies with the published cDNA sequence: a G→A substitution at bp 542 which converts the glycine to an aspartate codon, and a C→T change at bp 1032 which does not alter the amino acid (threonine). Since all DNAs tested (>50 chromosomes) carried the A at bp 542 and the T at bp 1032, it is likely that the sequence of the original cDNA contained some cloning artifacts.

Genotype:phenotype correlation

Table 2 summarizes the current status of mutations in severe MTHFR deficiency. In 8 patients, both mutations have been identified; in 5 patients (3 families), only 1 mutation has been identified. Overall the correlation between the genotype, enzyme activity, and phenotype is quite consistent. Five patients, with onset of symptoms within the first year of life, had ≦3% of control activity. Three of these patients had missense mutations in the homozygous state: two patients with the threonine to methionine substitution (C692T) and one patient with the proline to leucine substitution (C764T). The nonsense mutation (C559T) in the homozygous state in patients 1554 and 1627 (previously reported in Goyette P et al., Nature Genetics, 1994, 7:195-200) is also associated with a neonatal severe form, as expected.

The other patients in Table 2 had ≧6% of control activity and onset of symptoms within or after the 2nd decade of life; the only exception is patient 1834, as previously reported (Goyette P et al., Nature Genetics, 1994, 7:195-200). The three patients (356, 458 and 1396) with missense mutations (G167A, C985T, C1015T and C1081T) are similar to those previously reported (patients 1779, 1834 and 1863) who had an arginine to glutamine substitution and a second unidentified mutation (Goyette P et al., Nature Genetics, 1994, 7:195-200). The sisters with the 5′ splice mutation and an unidentified second mutation also had levels of activity in the same range and onset of symptoms in the second decade, but the activity is likely due to the second unidentified allele.

Discussion

The patients come from diverse ethnic backgrounds. Although patients 1554 and 1627 are both Native Americans, the mutations occur on different haplotypes, suggesting recurrent mutation rather than identity by descent. Since the substitution occurs in a CpG dinucleotide, a “hot spot” for mutation, recurrent mutation is a reasonable hypothesis. It is difficult to assess whether some mutations are population-specific since the numbers are too small at the present time.

MTHFR deficiency is the most common inborn error of folate metabolism, and a major cause of hereditary homocysteinemia. The recent isolation of a cDNA for MTHFR has permitted mutational analysis at this locus, with the aims of defining important domains for the enzyme and of correlating genotype with phenotype in MTHFR-deficient patients.

The 7 mutations described here (6 single amino acid substitutions and a 5′ splice site mutation) bring the total to 9 mutations identified thus far in severe MTHFR deficiency and complete the mutation analysis for 8 patients. The identification of each mutation in only one or two families points to the striking degree of genetic heterogeneity at this locus. Seven of the 9 mutations are located in CpG dinucleotides, which are prone to mutational events.

5′ splice site mutation

The G→A substitution at the GT dinucleotide of the 5′ splice site in patients 354 and 355 results in a 57 bp in-frame deletion of the coding sequence, which should delete 19 amino acids of the protein. This deletion occurs as a result of the activation of a cryptic 5′ splice site (AG/gc) even though this cryptic site does not have a perfect 5′ splice site consensus sequence (AG/gt). However, GC (instead of GT) as the first 2 nucleotides of an intron has been reported in several naturally-occurring splice sites, such as in the genes for human prothrombin and human adenine phosphoribosyltransferase and twice within the gene for the largest subunit of mouse RNA polymerase II. The remaining nucleotides of the cryptic site conform to a normal splice site consensus sequence with its expected variations (A₆₀ G₇₉/g₁₀₀t₁₀₀a₅₉a₇₁g₈₂t₅₀). It is unlikely that the deleted enzyme resulting from this aberrantly-spliced mRNA would have any activity; 8 of the 19 deleted amino acids are conserved in the E. Coli enzyme. Although the 2 patients show residual enzyme activity in the range of 20% of controls, the activity is probably due to the unidentified second allele in these patients.

6 missense mutations

The Arg→Cys substitution (C1081T) in patient 1396 is within a hydrophilic sequence previously postulated to be the linker region between the catalytic and regulatory domains of MTHFR (Goyette P et al., Nature Genetics, 1994, 7:195-200). These 2 domains are readily separable by mild trypsinization of the porcine enzyme. The linker domain, a highly-charged region, is likely to be located on the outside surface of the protein and therefore more accessible to proteolysis. Because the Arg→Cys substitution converts a charged hydrophilic residue to an uncharged polar residue, it cannot be considered a conservative change, and could affect the stability of the enzyme.

The 2 Arg→Cys substitutions identified in patients 356 and 458 (C985T and C1015T, respectively) may be involved in binding the FAD cofactor. Previous work in the literature showed that heating fibroblast extracts at 55°, in the absence of the FAD cofactor, inactivated MTHFR completely. The addition of FAD to the reaction mixture before heat inactivation restored some enzyme activity to control extracts and to extracts from some patients, while the extracts of patients 356 and 458 were unaffected. Based on these observations, it was suggested that these 2 patients had mutations affecting a region of the protein involved in binding FAD. The 2 mutations are found in close proximity to each other, within 11 amino acids. In patient 356, the Arg residue is evolutionarily-conserved in E. Coli and is found in a stretch of 9 conserved amino acids, suggesting a critical role for this residue; the altered Arg residue in patient 458 is not evolutionarily-conserved. Crystal structure analysis of medium chain acyl-CoA dehydrogenase (MCAD), a flavoprotein, has defined critical residues involved in the binding of FAD. Two consecutive residues of the MCAD protein, Met165 and Trp166, involved in interactions with FAD, can also be identified in MTHFR, 3 and 4 amino acids downstream, respectively, from the Arg residue altered in patient 458.

The Thr→Met substitution (C692T) is found in a region of high conservation with the E. Coli enzyme and in a region of good homology with human dihydrofolate reductase (DHFR) (FIG. 11). In FIG. 11,=is identity; • is homology; and $ is identity to bovine DHFR enzyme. An asterisk (*) indicates location of Thr→Met substitution. Considering the early-onset phenotype of the patients, one can assume that the threonine residue is critical for activity or that it contributes to an important domain of the protein. This region of homology in DHFR contains a residue, Thr136, which has been reported to be involved in folate binding. This Thr residue in DHFR aligns with a Ser residue in MTHFR, an amino acid with similar biochemical properties. The Thr→Met substitution is located 8 amino acids downstream from this Ser codon, in the center of the region of homology between the 2 enzymes. It is therefore hypothesized that the Thr→Met substitution may alter the binding of the folate substrate.

The G167A (Arg→Gln) and C764T (Pro→Leu) substitutions both affect non-conserved amino acids. Their importance in the development of MTHFR deficiency cannot be determined at the present time. All the mutations identified thus far are located in the 5′ end of the coding sequence, the region thought to encode the catalytic domain of MTHFR. Mutation analysis has been useful in beginning to address the structure: function properties of the enzyme as well as to understand the diverse phenotypes in severe MTHFR deficiency.

III. Identification of A→V mutation

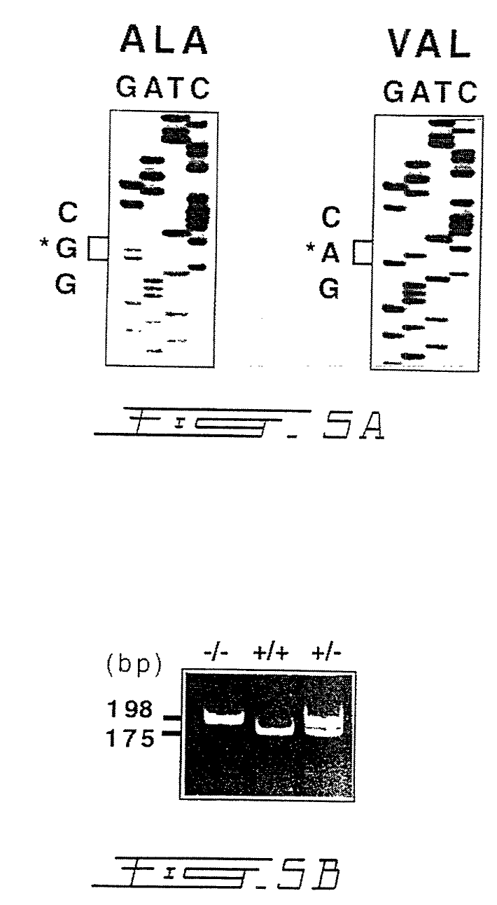

SSCP analysis and direct sequencing of PCR fragments were used to identify a C to T substitution at bp 677, which converts an alanine residue to a valine residue (FIG. 5A). The primers for analysis of the A→V change are: 5′-TGAAGGAGAA GGTGTCTGCG GGA-3′ (SEQ ID NO:13) (exonic) and 5′-AGGACGGTGC GGTGAGAGTG-3′ (SEQ ID NO:14) (intronic); these primers generate a fragment of 198 bp. FIG. 5A depicts the sequence of two individuals, a homozygote for the alanine residue and a homozygote for the valine residue. The antisense strands are depicted. This alteration creates a HinfI site (FIG. 5B), which was used to screen 114 unselected French Canadian chromosomes; the allele frequency of the substitution was 0.38. The substitution creates a HinfI recognition sequence which digests the 198 bp fragment into a 175 bp and a 23 bp fragment; the latter fragment has been run off the gel. FIG. 5B depicts the three possible genotypes. The frequency of the 3 genotypes were as follows: −/−, 37%; +/−, 51%; and +/+, 12% (the (+) indicates the presence of the HinfI restriction site and a valine residue).

The alanine residue is conserved in porcine MTHFR, as well as in the corresponding metF and stymetF genes of E. Coli and S. Typhimurium, respectively. The strong degree of conservation of this residue, and its location in a region of high homology with the bacterial enzymes, alluded to its importance in enzyme structure or function. Furthermore, the frequency of the (+/+) genotype was consistent with the frequency of the thermolabile MTHFR variant implicated in vascular disease.

Clinical material

To determine the frequency of the A→V mutation, DNA from 57 individuals from Quebec was analyzed by PCR and restriction digestion. The individuals, who were all French Canadian, were not examined clinically or biochemically.

The 40 individuals analyzed in Table 3 had been previously described in Engbersen et al. (Am. J. Hum. Genet., 1995, 56:142-150). Of the 13 cardiovascular patients, 8 had cerebrovascular arteriosclerosis and 5 had peripheral arteriosclerosis. Five had thermolabile MTHFR while 8 had thermostable MTHFR (greater than 33% residual activity after heating). Controls and patients were all Dutch-Caucasian, between 20-60 years of age. None of these individuals used vitamins which could alter homocysteine levels. Enzyme assays and homocysteine determinations were also reported by Engbersen et al. (Am. J. Hum. Genet., 1995, 56:142-150).

TABLE 3
Correlation between MTHFR genotype and enzyme activity,
thermolability and plasma homocysteine level

| | −/− | +/− | +/+ |
|---|---|---|---|
| | n = 19 | n = 9 | n = 12 |
| specific activity^{a,b} | 22.9 ± 1.7 | 15.0 ± 0.8 | 6.9 ± 0.6 |
| (nmol CH₂0/ | (11.8-33.8) | (10.2-18.8) | (2.6-10.2) |
| mg · protein/hr) | | | |
| residual activity after | 66.8 ± 1.5 | 56.2 ± 2.8 | 21.8 ± 2.8 |
| heating^{a,b} (%) | (55-76) | (41-67) | (10-35) |
| plasma homocysteine^{a,c} | 12.6 ± 1.1 | 13.8 ± 1.0 | 22.4 ± 2.9 |
| (μM)(after fasting) | (7-21) | (9.6-20) | (9.6-42) |
| plasma homocysteine^{a,c} | 413 ± 5.0^{d} | 41 ± 2.8 | 72.6 ± 11.7^{e} |
| (μM)(post-methionine load) | (20.9-110) | (29.1-54) | (24.4-159) |

^{a}one-way anova p < .01
^{b}paired t test for all combinations p < .01
^{c}paired t test p < .05 for +/+ group versus +/− group or −/− group; p > .05 for +/− versus −/− group.
^{d}n = 18 for this parameter
^{e}n = 11 for this parameter

Enzyme activity and plasma homocysteine were determined as previously reported. Each value represents mean±standard error. The range is given in parentheses below the mean.

Correlation of A→V mutation with altered MTHFR function

A genotypic analysis was performed and enzyme activity and thermolability were measured in a total of 40 lymphocyte pellets from patients with premature vascular disease and controls. 13 vascular patients were selected from a previous study (Engbersen et al., Am. J. Hum. Genet., 1995, 56:142-150), among which 5 were considered to have thermolabile MTHFR. From a large reference group of 89 controls, all 7 individuals who had thermolabile MTHFR were studied, and an additional 20 controls with normal MTHFR were selected from the same reference group. Table 3 documents the relationship between genotypes and specific enzyme activity, thermolability and plasma homocysteine level. The mean MTHFR activity for individuals homozygous for the substitution (+/+) was approximately 30% of the mean activity for (−/−) individuals, homozygous for the alanine residue. Heterozygotes had a mean MTHFR activity that was 65% of the activity of (−/−) individuals; this value is intermediate between the values for (−/−) and (+/+) individuals. The ranges of activities showed some overlap for the heterozygous and (−/−) genotypes, but homozygous (+/+) individuals showed virtually no overlap with the former groups. A one-way analysis of variance yielded a p value<0.0001; a pairwise Bonferroni t test showed that all three genotypes were significantly different with p<0.01 for the three possible combinations.

The three genotypes were all significantly different (p<0.0l) with respect to enzyme thermolability. The mean residual activity after heat inactivation for 5 minutes at 46° was 67%, 56% and 22% for the (−/−), (+/−) and (+/+) genotypes, respectively. While the degree of thermolability overlaps somewhat for (−/−) individuals and heterozygotes, individuals with two mutant alleles had a distinctly lower range. Every individual with the (+/+) genotype had residual activity <35% after heating, and specific activity <50% of that of the (−/−) genotype.

Total homocysteine concentrations, after fasting and 6 hours after methionine loading, were measured in plasma by high performance liquid chromatography using fluorescence detection. Fasting homocysteine levels in (+/+) individuals were almost twice the value for (+/−) and (−/−) individuals. The differences among genotypes for plasma homocysteine were maintained when homocysteine was measured following 6 hours of methionine loading. A one-way anova yielded a p<0.01 for the fasting and postmethionine homocysteine levels. A pairwise Bonferroni t test showed that only homozygous mutant individuals had significantly elevated homocysteine levels (p<0.05).

PCR-based mutagenesis for expression of A→V mutation in vitro

PCR-based mutagenesis, using the cDNA-containing Bluescript™ vector as template, was used to create the A to V mutation. Vent™ polymerase (NEB) was used to reduce PCR errors. The following primers were used: primer 1, bp −200 to −178, sense; primer 2, bp 667 to 687, antisense, containing a mismatch, A, at bp 677; primer 3, 667 to 687, sense, containing a mismatch, T, at bp 677; primer 4, bp 1092 to 1114, antisense. PCR was performed using primers 1 and 2 to generate a product of 887 bp, and using primers 3 and 4 to generate a product of 447 bp. The two PCR fragments were isolated from a 1.2% agarose gel by Geneclean™ (BIO 101). A final PCR reaction, using primers 1 and 4 and the first 2 PCR fragments as template, was performed to generate a 1.3 kb band containing the mutation. The 1.3 kb fragment was digested with NcoI and MscI, and inserted into the wild-type cDNA-containing expression vector by replacing the sequences between the NcoI site at bp 11 and the MscI site at bp 943. The entire replacement fragment and the cloning sites were sequenced to verify that no additional changes were introduced by PCR.

Expression analysis of wild-type and mutagenized cDNA

Overnight cultures of JM105™ containing vector alone, vector+wild-type MTHFR cDNA, or vector+mutagenized cDNA were grown at 37° C. in 2×YT media with 0.05 mg/ml ampicillin. Fresh 10 ml. cultures of each were inoculated with approximately 50 μL of overnight cultures for a starting O.D. of 0.05, and were grown at 37° C. to an O.D. of 1 at 420 nM. Cultures were then induced for 2 hrs. with 1 mM IPTG and pelleted. The cells were resuspended in TE buffer with 2 μg/ml aprotinin and 2 μg/ml leupeptin (3.5×wet weight of cells). Cell suspensions were sonicated on ice for 3×15 sec. to break open cell membranes and then centrifuged for 30 min. at 4° C. to pellet cell debris and unlysed cells. The supernatant was removed and assayed for protein concentration with the Bio-Rad™ protein assay. Western analysis was performed using the Amersham ECLTM kit according to the instructions of the supplier, using antiserum generated against purified porcine liver MTHFR. Enzymatic assays were performed by established procedures; pre-treating the extracts at 46° C. for 5 min. before determining activity assessed thermolability. Specific activities (nmol formaldehyde/hr./mg. protein) were calculated for the 2 cDNA-containing constructs after subtraction of the values obtained with vector alone (to subtract background E. Coli MTHFR activity).

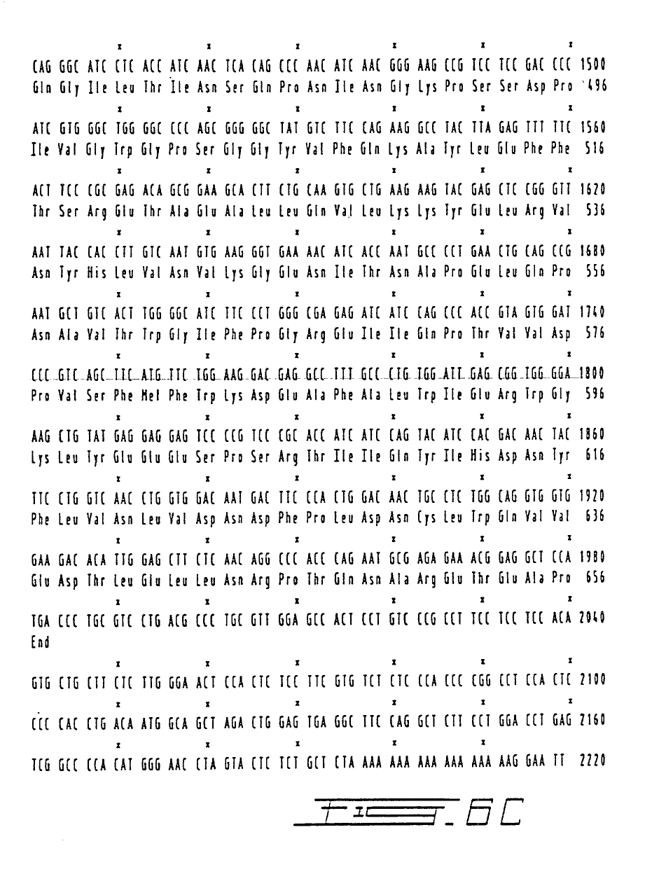

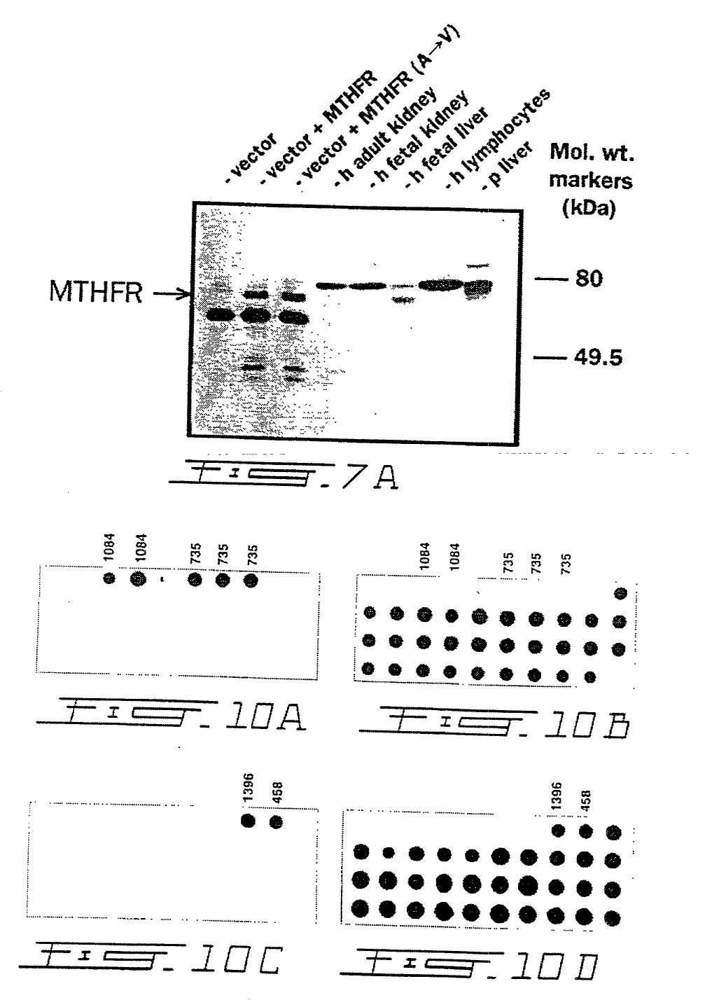

The MTHFR cDNA (2.2 kb) (FIG. 6) has an open reading frame of 1980 bp, predicting a protein of 74.6 kDa. The purified porcine liver enzyme has been shown to have subunits of 77 kDa. Western analysis (FIG. 7A) of several human tissues and of porcine liver has revealed a polypeptide of 77 kDa in all the studied tissues, as well as an additional polypeptide of approximately 70 kDa in human fetal liver and in porcine liver, suggesting the presence of isozymes. Two μg of bacterial extract protein was used for lanes 1-3. The tissues (lanes 4-8) were prepared by homogenization in 0.25M sucrose with protease inhibitors (2 μg/ml each of aprotinin and leupeptin), followed by sonication (3×15 sec.) on ice. The extracts were spun for 15 min. in a microcentrifuge at 14,000 g and 100 μg of supernatant protein was used for Western analysis. h=human; p=porcine.

The wild-type cDNA and a mutagenized cDNA, containing the A→V substitution, were expressed in E. Coli to yield a protein of approximately 70 kDa (FIG. 7A), which co-migrates with the smaller polypeptide mentioned above. Treatment of extracts at 46° C. for 5 minutes revealed that the enzyme containing the substitution was significantly more thermolabile than the wild-type enzyme (p<0.001; FIG. 7B). Two separate experiments (with 3-4 replicates for each construct for each experiment) were performed to measure thermostable activity of the wild-type MTHFR and mutagenized MTHFR A→V cDNAs. The values shown represent mean+standard error for each experiment, as % of residual activity after heating. The means of the specific activities before heating (expressed as nmol formaldehyde/hr./mg. protein) were as follows: Exp. 1, 3.8 and 5.3 for MTHFR and MTHFR A→V, respectively; Exp. 2, 6.2 and 7.5 for MTHFR and MTHFR A→V, respectively. The expression experiments were not designed to measure differences in specific activity before heating, since variation in efficiencies of expression could contribute to difficulties in interpretation. Curiously though, the specific activity for the mutant construct was higher in both experiments. It is possible that the mutant protein has increased stability in E. Coli, or that inclusion bodies in the extracts contributed to differences in recovery of properly-assembled enzyme.

These studies have identified a common substitution in the MTHFR gene which results in thermolability in vitro and in vivo. The mutation, in the heterozygous or homozygous state, correlates with reduced enzyme activity and increased thermolability of MTHFR in lymphocyte extracts. A significant elevation in plasma homocysteine was observed in individuals who were homozygous for the mutation. Statistically-significant differences for homocysteine levels were not observed between heterozygotes and (−/−) individuals; this observation is not surprising, since plasma homocysteine can be influenced by several environmental factors, including intake of folate, vitamin B₁₂, vitamin B₆, and methionine, as well as by genetic variation at other loci, such as the cystathionine-β-synthase gene.

The alanine to valine substitution conserves the hydrophobicity of the residue and is associated with small changes in activity, in contrast to non-conservative changes, such as the previously-reported arginine to glutamine change in MTHFR, which is associated with a greater decrease in enzyme activity and severe hyperhomocysteinemia. The alanine residue is situated in a region of homology with the bacterial metF genes. The same region of homology was also observed in the human dihydrofolate reductase (DHFR) gene (FIG. 11), although the alanine residue itself is not conserved; this region of amino acids 130-149 of DHFR contains T136 which has been implicated in folate binding in an analysis of the crystal structure of recombinant human DHFR. It is tempting to speculate that this region in MTHFR is also involved in folate binding and that the enzyme may be stabilized in the presence of folate. This hypothesis is compatible with the well-documented influence of folate on homocysteine levels and with the reported correction of mild hyperhomocysteinemia by folic acid in individuals with premature vascular disease, and in individuals with thermolabile MTHFR.

Although the cDNA is not long enough to encode the larger MTHFR polypeptide, it is capable of directing synthesis of the smaller isozyme. The ATG start codon for this polypeptide is within a good consensus sequence for translation initiation. Whether the isozyme is restricted to liver and what its role is in this tissue remain to be determined.

These data have identified a common genetic change in MTHFR which results in thermolability; these experiments do not directly address the relationship between this change and vascular disease. Nonetheless, this polymorphism represents a diagnostic test for evaluation of MTHFR thermolability in hyperhomocysteinemia. Large case-control studies are required to evaluate the frequency of this genetic change in various forms of occlusive arterial disease and to examine the interaction between this genetic marker and dietary factors. Well-defined populations need to be examined, since the limited data set thus far suggests that population-specific allele frequencies may exist. More importantly, however, the identification of a candidate genetic risk factor for vascular disease, which may be influenced by nutrient intake, represents a critical step in the design of appropriate therapies for the homocysteinemic form of arteriosclerosis.

cDNA FOR MTHFR AND ITS POTENTIAL UTILITY

The cDNA sequence is a necessary starting point for the detection of MTHFR sequence abnormalities that would identify individuals at risk for cardiovascular and neurological diseases, as well as other disorders affected by folic acid metabolism. Diagnostic tests by DNA analysis are more efficient and accurate than testing by enzymatic/biochemical assays. Less blood is required and results are available in a shorter period of time. The tests could be performed as a routine operation in any laboratory that performs molecular genetic diagnosis, without the specialized reagents/expertise that is required for an enzyme-based test.

The second major utility of the cDNA would be in the design of therapeutic protocols, for correction of MTHFR deficiency. These protocols could directly involve the gene, as in gene therapy trials or in the use of reagents that could modify gene expression. Alternatively, the therapy might require knowledge of the amino acid sequence (derived from the cDNA sequence), as in the use of reagents that would modify enzyme activity. The identification of sequences and/or sequence changes in specific regions of the cDNA or protein, such as FAD binding sites or folate-binding sites, are useful in designing therapeutic protocols involving the above nutrients.

UTILITY OF INVENTION IN CLINICAL AND DIAGNOSTIC STUDIES

Coronary artery disease patients in Montreal (n=153) were studied to examine the frequency of the alanine to valine substitution. Fourteen percent of the patients were homozygous for this mutation. An analysis of 70 control individuals (free of cardiovascular disease) demonstrated that only seven % of these individuals were homozygous for the alanine to valine mutation.

Analysis of homocysteine levels in 123 men of the above patient group indicated that the mutant allele significantly raised homocysteine levels from 10.2 micromoles/L in homozygous normal men to 11.5 and 12.7 in heterozygotes and homozygous mutants, respectively.

Families with a child with spina bifida, a neural tube defect, have been examined for the presence of the alanine to valine mutation. Approximately 16% of mothers who had a child with spina bifida were homozygous for this mutation, while only 5% of control individuals were homozygous. Fathers of children with spina bifida also had an increased prevalence of the homozygous mutant genotype (10%) as did the affected children themselves (13%).

Table 4 indicates the interactive effect of folic acid with the homozygous mutant alanine to valine change. In a study of families from Framingham, Mass. and Utah, individuals who were homozygous mutant but had folate levels above 5 ng/ml did not have increased homocysteine levels compared to individuals with the normal or heterozygous genotype. However, individuals who were homozygous mutant but had folate levels below 5 ng/ml had homocysteine levels that were significantly higher than the other genotypes.

TABLE 4
Mean fasting and PML homocysteine levels for different
MTHFR genotypes

| | MTHFR genotype | | | |
|---|---|---|---|---|
| Plasma | Normals | Heterozygotes | Homozygotes | |
| Homocysteine | (−/−) | (+/−) | (+/+) | P_{trend} |
| N | 58 | 61 | 30 | |
| Fasting* | 9.4 | 9.2 | 12.1 | 0.02 |
| Folate < 5 ng/mL | 10.2 | 10.4 | 15.2 | 0.002 |
| Folate³ 5 ng/mL | 8.2 | 7.5 | 7.5 | 0.52 |
| Post-Methionine | 30.0 | 30.9 | 31.3 | 0.62 |
| load | | | | |

*Significant interaction between folate levels and genotype (p = 0.03)

Table 4 provides preliminary data for therapeutic intervention by folic acid supplementation to individuals who are homozygous for the alanine to valine change. The data suggest that higher levels of plasma folate would lead to normalization of homocysteine levels in mutant individuals and might prevent the occurrence of disorders associated with high homocysteine levels, such as cardiovascular disease, neural tube defects, and possibly other disorders. Folic acid supplementation for mutant individuals might also restore methionine and S-adenosylmethionine levels to normal. This would be relevant for disorders that are influenced by methylation, such as neoplasias, developmental anomalies, neurologic disease, etc.

Genetic polymorphism in methylenetetrahydrofolate reductase (MTHFR) associated with decreased activity

A common mutation (C677T) results in a thermolabile enzyme with reduced specific activity (approximately 35% of control values in homozygous mutant individuals). Homozygous mutant individuals (approximately 10% of North Americans) are predisposed to mild hyperhomocysteinemia, when their folate status is low. This genetic-nutrient interactive effect is believed to increase the risk for neural tube defects (NTD) and vascular disease. There has been reported an increased risk for spina bifida in children with the homozygous mutant genotype for C677T. With the present invention, a second common variant in MTHFR (A1298C), an E to A substitution has been characterized. Homozygosity was observed in approximately 10% of Canadian individuals. This polymorphism was associated with decreased enzyme activity; homozygotes had approximately 60% of control activity in lymphocytes.

A sequence change (C1298A) has been identified. Heterozygotes for both the C677T and the A1298C mutation, approximately 15% of individuals, had 50%-60% of control activity, a value that was lower than that seen in single heterozygotes for the C677T variant. No individuals were homozygous for both mutations. A silent genetic variant T1317C, was identified in the same exon. It was relatively infrequent (allele frequency=5%) in the study group, but was common in a small sample of African individuals (allele frequency=39%).

In addition, by virtue of the role of MTHFR in folate-dependent homocysteine metabolism, the C677T mutation predisposes to mild hyperhomocysteinemia, a risk factor for vascular disease, in the presence of low folate status. By the present invention, the frequency of the A1298C variant has been determined and its potential impact on enzyme function has been assessed.

Patients with spina bifida and mothers of patients were recruited from the Spina Bifida Clinic at the Montreal Children's Hospital following approval from the Institutional Review Board. Control children and mothers of controls were recruited from the same institution. Blood samples were used to prepare DNA from peripheral leukocytes, to assay MTHFR activity in lymphocyte extracts, and to measure total plasma homocysteine (tHcy). The presence of the C677T mutation (A to V) was evaluated by PCR and HinfI digestion (2). The A1298C mutation was initially examined by PCR and MboII digestion (5). The silent mutation, T1317C, was identified by SSCP and sequence analysis in a patient with severe MTHFR deficiency and homocystinuria. This patient, an African-American female, already carries a previously-described splice mutation (patient 354 (8)). Since this mutation also creates a MboII site and results in a digestion pattern identical to that of the A1298C mutation, distinct artificially-created restriction sites were used to distinguish between these 2 mutations. Detection of the A1298C polymorphism was performed with the use of the sense primer 5′-GGGAGGAGCTGACCAGTGCAG-3′ and the antisense primer (5′-GGGGTCAGGCCAGGGGCAG-3′), such that the 138bp PCR fragment was digested into 119bp and 19bp fragments by Fnu4HI in the presence of the C allele. An antisense primer (5′-GGTTCTCCCGAGAGGTAAAGATC-3′), which introduces a TaqI site, was similarly designed to identify the C allele of the T1317C polymorphism. Together with a sense primer (5′-CTGGGGATGTGGTGGCACTGC-3′), the 227bp fragment is digested into 202bp and 25bp fragments.

TABLE 5
Genotype distributions, MTHFR activity (nmol
formaldehyde/mg protein/hour), and total plasma
homocysteine (tHcy;μM) for mothers and children

| E/E | | | E/A | | | A/A | | |
|---|---|---|---|---|---|---|---|---|
| A/A | A/V | V/V | A/A | A/V | V/V | A/A | A/V | V/V |

Mothers (n = 141)

| | | | | | | | | | |
|---|---|---|---|---|---|---|---|---|---|
| # | 24 | 32 | 19 | 27 | 26 | 0 | 13 | 0 | 0 |
| % | 17 | 23 | 13 | 19 | 18 | 0 | 9 | 0 | 0 |
| MTHFR | 49.0 ± 18.9 | 33.0 ± 10.8 | 15.7 ± 4.5 | 45.0 ± 16.0 | 30.2 ± 19.3 | — | 32.1 ± 9.0 | — | — |
| | [14] | [19]* | [11]* | [15] | [15] | | [7]* | | |
| THcy | 9.5 ± 3.1 | 10.0 ± 3.2 | 12.2 ± 7.1 | 8.4 ± 2.1 | 10.0 ± 3.1 | — | 9.5± 2.0 | — | — |
| | [24] | [32] | [19]** | [25] | [26] | | [13] | | |

Children (n = 133)

| | | | | | | | | | |
|---|---|---|---|---|---|---|---|---|---|
| # | 23 | 43 | 18 | 20 | 15 | 1 | 13 | 0 | 0 |
| % | 17 | 32 | 13 | 15 | 11 | 1 | 10 | 0 | 0 |
| MTHFR | 52.0 ± 17.0 | 38.2 ± 15.0 | 16.2 ± 5.3 | 35.7 ± 9.7 | 26.1 ± 5.0 | 21.6 | 29.5 ± 10.3 | — | — |
| | [12] | [27]* | [11]* | [18]* | [9]* | [1] | [6]* | | |
| THcy | 7.6 ± 2.5 | 8.2 ± 3.0 | 9.7± 5.1 | 7.5 ± 2.3 | 8.1 ± 2.8 | 9.5 | 7.4 ± 1.5 | — | — |
| | [23] | [43] | [18]** | [20] | [15] | [1] | [13] | | |

The three A1298C genotypes and the three C677T genotypes are designated by the amino acid codes: EE, EA, AA, and AA, AV, VV, respectively. Statistical significance was assessed by student t-test, in comparison with EEAA values.
*(p < 0.05);
**(p < 0.07).
Standard deviations are given and square brackets indicate the number of individuals for whom MTHFR activities and homocysteine levels were available.

The frequencies of the three genotypes for the A1298C mutation (EE, EA and AA) were not different between case and control mothers, or between case and control children (data not shown). Consequently, all the mothers and all the children were grouped together for analyses (Table 5). Nine % of mothers had the homozygous AA genotype while 37% were heterozygous. This frequency is quite similar to the frequency of the homozygous mutant genotype (VV) for the C677T polymorphism. In the MTHFR human cDNA sequence mentioned above, the cDNA contained the C nucleotide at bp 1298 change as a C1298A substitution. Since the A nucleotide is clearly the more frequent base at this position, the A1298C nomenclature was chosen.

Since the C677T mutation (A to V) decreases MTHFR activity and increases homocysteine levels, the three genotype groups for the A1298C (E to A) mutation were further stratified by the genotype for the A to V mutation, to avoid the confounding influence of the latter polymorphism on MTHFR activity and homocysteine levels. The frequencies of the 9 genotypes, with MTHFR activity and homocysteine levels for each genotype, are shown in Table 5. If the mothers and children without either mutation i.e. EE/AA are used as the reference (control) group, the mothers and children that are homozygous for the A1298C mutation (AAAA) have approximately 65% and 57%, respectively, of control MTHFR activity. Heterozygotes for the C677T change alone (EEAV) have approximately 70% of control activity, as reported in other studies, while double heterozygotes (EAAV), 18% of mothers and 11% of children, have an additional loss of activity (approximately 62% and 50% of control values, respectively).

Homocysteine levels were not significantly increased by the A1298C mutation, but homocysteine was elevated (with borderline significance, p≦0.07) in mothers and children who were homozygous for the C677T change. The small number of individuals who were homozygous for the A1298C mutation (n=13) may have influenced the power of the statistical analyses and precluded an investigation of the genetic-nutrient interactive effect that leads to mild hyperhomocysteinemia, as seen in individuals with the C677T mutation.

The T1317C substitution does not alter the amino acid (phenylalanine) and is likely a benign change, although a splicing defect cannot be ruled out at the present time. In an evaluation of 38 control mothers from this study, 2 were found to be heterozygous and one was identified as a homozygote, resulting in an allele frequency of 5% (4/76). Since this substitution was identified in an African-American female, control African individuals were also examined (n=9). Seven of these were heterozygous, resulting in an allele frequency of 39% (7/18).

The A1298C mutation clearly reduces MTHFR activity, albeit to a lesser extent than the C677T mutation. Consequently its effect on homocysteine levels is also attenuated and, in fact, may only be significant when an individual carries both mutations and/or has poor nutrient status. However, since double heterozygotes are estimated to represent approximately 15% of the population, this variant should be examined in conjunction with the C677T variant in studies of hyperhomocysteinemia.

The A1298C mutation is clearly polymorphic in Canadian individuals and should be examined in other populations. The A nucleotide is likely to be the ancestral sequence since it represents the more common allele, although the original human MTHFR cDNA sequence (GenBank accession number U09806) carried the C nucleotide. This polymorphism is similar in frequency to the C677T polymorphism. Presumably the two substitutions arose separately on a A1298/C677 or E/A haplotype, since the haplotype with both substitutions (C1298/T677 or A/V) is extremely rare. One such haplotype was seen in a child with the EAVV genotype, suggesting a recombinant chromosome.

Doubly homozygous individuals (AAVV) were not observed in this study. Since the double mutation in cis is rare, it is possible that not enough alleles were studied. Larger studies in other populations might result in the identification of these individuals. Presumably the MTHFR activity would be even lower and homocysteine levels might be higher than those observed thus far.

The C677T polymorphism in exon 4 is within the N-terminal catalytic domain of the enzyme whereas the A1298C polymorphism in exon 7 is within the C-terminal regulatory domain. The more dramatic effect on enzyme activity with the first polymorphism may be a consequence of its location within the catalytic region. The second polymorphism could affect enzyme regulation, possibly by S-adenosylmethionine, an allosteric inhibitor of MTHFR, which is known to bind in the C-terminal region.

Many studies have examined the effects of the C677T polymorphism on MTHFR enzyme activity and on homocysteine levels. Although the correlation between the presence of this substitution and decreased enzyme activity/increased homocysteine levels has been quite good, the variability in results, particularly in heterozygous individuals, may reflect the presence of a second common variant in the population.

The third variant, T1317C, was present on 5% of alleles in Canadian individuals but appears to be extremely common in individuals of African ancestry. The methodology outlined in this report should be used to assess the frequency of the A1298C and T1317C in other populations, since the use of the MboII restriction site for analysis of the A1298C change, as first reported, would not discriminate between the 2 polymorphisms.

The C677T mutation is a risk factor for hyperhomocysteinemia and has been implicated in both neural tube defects and vascular disease.

Gene structure of human and mouse methylenetetrahydrofolate reductase (MTHFR)

A human cDNA for MTHFR, 2.2 kb in length, has been expressed and shown to result in a catalytically-active enzyme of approximately 70 kDa. Fifteen mutations have been identified in the MTHFR gene: 14 rare mutations associated with severe enzymatic deficiency and one common variant associated with a milder deficiency. The common polymorphism has been implicated in three multifactorial diseases: occlusive vascular disease, neural tube defects and colon cancer. The human gene has been mapped to chromosomal region 1p36.3 while the mouse gene has been localized to distal Chromosome 4. The isolation and characterization of the human and mouse genes for MTHFR is herein reported. A human genomic clone (17 kb) was found to contain the entire cDNA sequence of 2.2 kb; there were 11 exons ranging in size from 102 bp to 432 bp. Intron sizes ranged from 250 bp to 1.5 kb with one exception of 4.2 kb. The mouse genomic clones (19 kb) start 7 kb 5′ to exon 1 and extend to the end of the coding sequence. The mouse amino acid sequence is approximately 90% identical to the corresponding human sequence. The exon sizes, locations of intronic boundaries, and intron sizes are also quite similar between the two species. The availability of human genomic clones has been useful in designing primers for exon amplification and mutation detection. The mouse genomic clones may be used to make constructs for gene targeting and generation of mouse models for MTHFR deficiency.

A common polymorphism, C677T has been identified, which converts an alanine codon to valine (Frosst et al., 1995). This common polymorphism, which is present on approximately 35% of alleles in the North American population, encodes the thermolabile variant and predisposes to mild hyperhomocysteinemia when folate status is low (Frosst et al., 1995; Jacques et al., 1996; Christensen et al., 1997). This genetic-nutrient interactive effect is believed to be a risk factor for arteriosclerosis (Frosst et al, 1995) and neural tube defects. In contrast, the mutant homozygous genotype may decrease the risk for colon cancer.

The characterization of the genomic structure for human MTHFR is reported herein. The corresponding analysis of the mouse gene, with a comparison of the overall organization of the gene and the amino acid sequences in these two species, is also shown.

Screening of genomic libraries

Genomic libraries were screened using standard methods of plaque hybridization. The 2.2 kb human cDNA was radiolabelled and used as a probe in screening both human and murine genomic libraries. Screening for the human gene was performed on a phage library of partial EcoRI digestion fragments from total genomic DNA (ATCC # 37385), and on a phage library of chromosome 1-specific complete EcoRI digestion fragments (ATCC# 57738). Screening for the mouse gene was performed on a KDASH library of partial Sau3A digestion fragments from total genomic DNA of mouse strain 129SV (obtained from Dr. J. Rossant, University of Toronto). Positive clones were purified by sequential rounds of screening and isolation, and phage DNA was isolated using phage DNA isolation columns (QIAGEN). Human clones were digested with EcoRI to release the inserts, and then with XbaI to facilitate cloning into Bluescript plasmid (Stratagene). The mouse clones were digested with SalI or EcoRI, and the inserts were subcloned into Bluescript.

Characterization of mouse CDNA sequences

Mouse genomic clones were sequenced (Sequenase kit, Amersham) using human cDNA primers spanning most of the available 2.2 kb cDNA. These sequences were then used to generate mouse-specific cDNA primers. The mouse-specific primers were used in PCR amplification of overlapping cDNA fragments from reverse-transcribed mouse liver RNA. The PCR products were subcloned into the PCRII vector (Invitrogen) and sequenced. Two different species of mouse (C57B1/6J and ct) were used to generate MTHFR sequence by RT-PCR, to ensure that the PCR protocol did not generate sequencing errors.

Characterization of intron boundaries and sizes, and restriction analysis of human and mouse genes

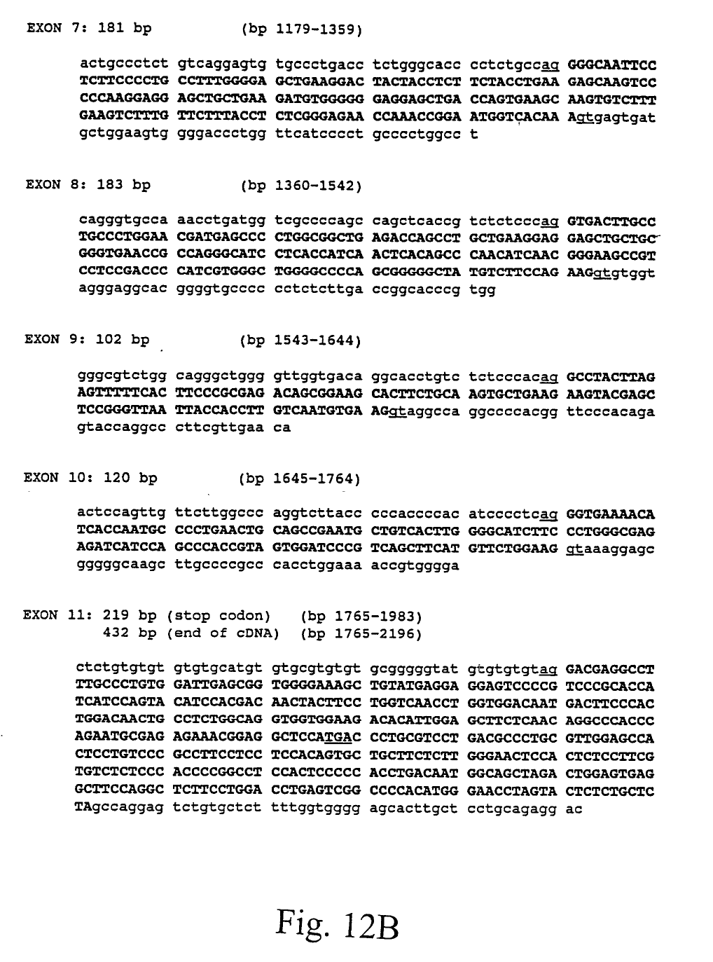

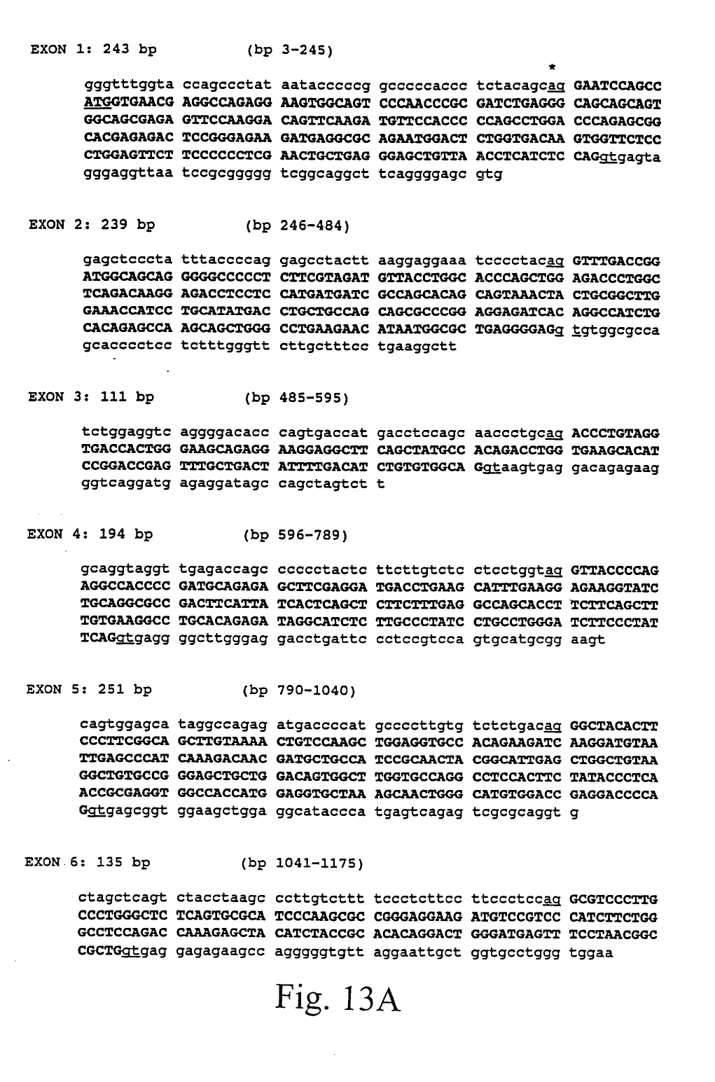

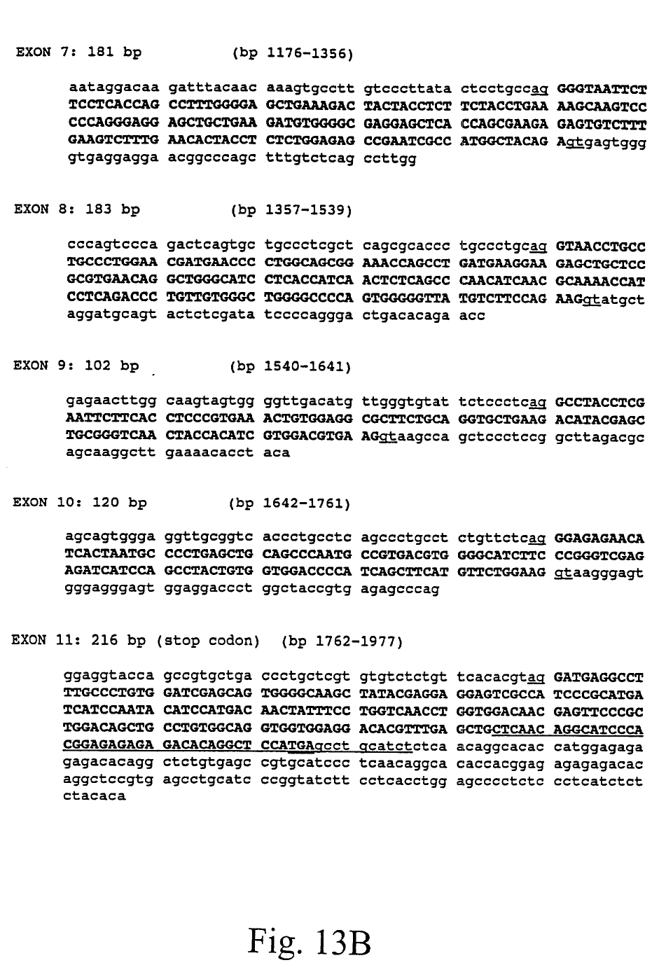

Primers from cDNA sequences of human and mouse were used to sequence the respective genomic clones. Intron boundaries were determined from regions of divergence between cDNA and genomic clone sequences, and by the identification of splice acceptor and donor consensus sites. Intronic sequences were obtained for 40-50 bp from the junctions and are shown in FIGS. 12A-12B (human) and FIGS. 13A-13B (mouse). The same cDNA primers were used in PCR amplification of total genomic DNA and of genomic clones to determine the approximate sizes of introns in the human and mouse genes. Table 6 lists the locations and approximate sizes of introns for both species. The PCR products were analyzed by restriction enzyme digestion to generate a preliminary restriction map of the gene. This restriction map was then confirmed by restriction analysis of the genomic clones in Bluescript.

Referring to FIGS. 12A-12B, the bp location of the exons within the cDNA, in parentheses, is based on the published human cDNA sequence (GenBank accession number U09806). Bp 1 is 12 bp upstream from the ATG in the original cDNA; an asterisk indicates the equivalent base here. Exon 1 contains the ATG start site (underlined), and exon 11 contains the termination codon (underlined). Uppercase characters indicate exonic sequences, and lower case characters are intronic. Consensus splice junction sequences are underlined. The 3′ boundary of exon 11 has been designated by the location of the polyA tail.

Referring to FIGS. 13A-13B, the bp location of the exons within the cDNA, in parentheses, is based on the equivalent bp 1 of the human sequences in FIGS. 12A-12B (bp 1 is indicated by an asterisk). Exon 1 contains the ATG start site (underlined), and exon 11 contains the termination codon (doubly underlined). Uppercase characters indicate exonic sequences, and lower case characters are intronic. Consensus splice junction sequences are underlined. The 3′ boundary of exon 11 is designated as the termination codon, since the site of polyadenylation is unknown. Also underlined in exon 11 is the first repeat of the 52 bp repeated element.

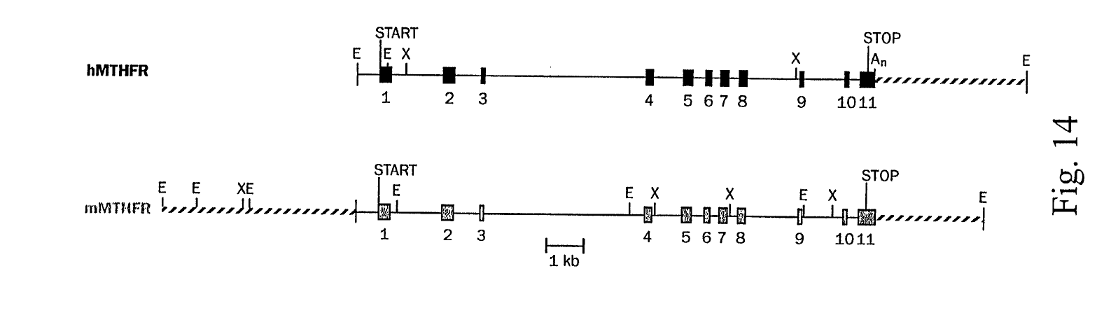

Referring to FIG. 14, exon sizes for human and mouse are reported in FIGS. 12A-12B and FIGS. 13A-13B, respectively. Exons are indicated in shaded boxes. Uncharacterized regions of the gene are hatched, and exon numbering corresponds to FIGS. 12A-12B and 13A-13B. E=EcoRI; X=XbaI; Aₙ=polyadenylation site. The EcoRI restriction site at the 5′ end of the mouse gene is part of the phage polylinker sequence.

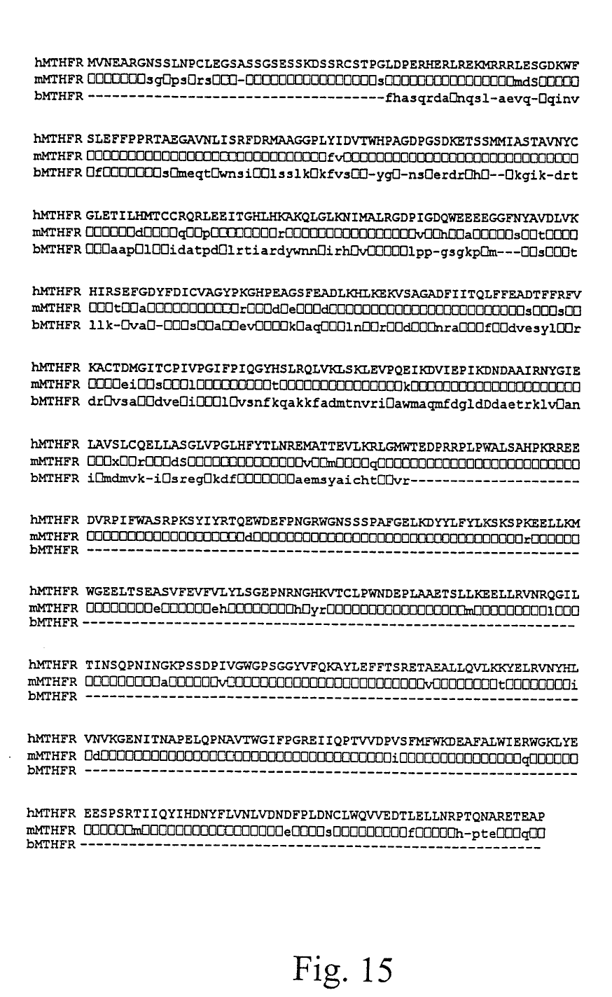

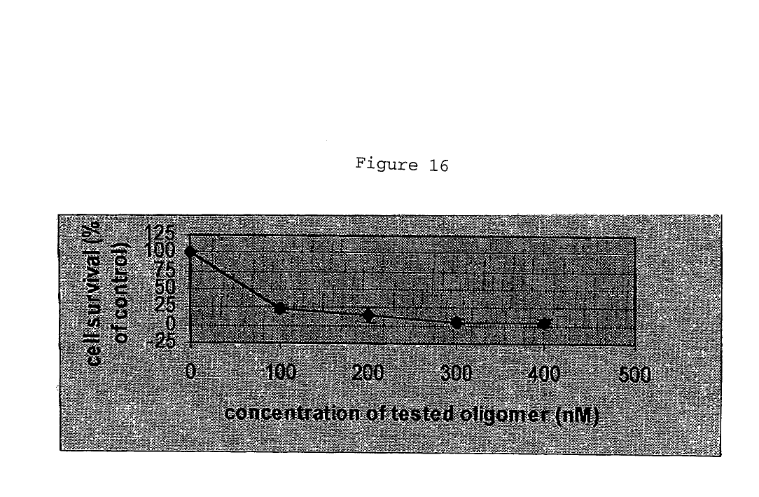

Referring to FIG. 15, residues that are identical to the human MTHFR sequence are shown as empty boxes, and gaps in amino acid homology are represented by a dash.

Human genomic clones

The genomic clones isolated from the human libraries contained a 16 kb EcoRI fragment, encompassing part of exon 1 and exons 2 through 11, and a 1 kb EcoRI fragment containing most of exon 1. Exon 1 is defined as the most 5′ exon from the previously published cDNA sequence; it contains the ATG start site that was used to express the human cDNA in bacterial extracts (Frosst et al. 1995). A graphic representation of the human gene and its restriction map are depicted in FIG. 3. The sequences of each exon and 50 bp of flanking intronic sequences are shown in FIGS. 12A-12B. Exons range in size from 102 bp to 432 bp, and the critical dinucleotides in the 5′ and 3′ splice site consensus sequences (GT and AG, respectively) are underlined. The 3′ boundary of exon 11 is defined by the site of polyadenylation in the cDNA; a possible polyadenylation signal (AACCTA) is present 15 bp upstream of the polyadenylation site, although it varies from the consensus sequence. Table 6 lists the locations and approximate sizes of introns as determined by PCR amplification; the introns range in size from approximately 250 bp to 4.2 kb.

Mouse genomic clones

Genomic clones isolated from the mouse libraries were digested with EcoRI for subcloning and characterization. Exon nomenclature is based on the corresponding human gene sequences. FIGS. 13A-13B list all known exons and their sizes, with 40 to 50 bp of flanking intronic sequences. FIG. 14 shows a graphic representation of the mouse genomic structure aligned with the human gene. The size of exon 11 is undetermined, since the sequence for this region was determined directly from the genomic clones. The termination codon is located within a region of 52 bp which is repeated 3 times in the gene. The significance of this, if any, is unknown at the present time. The dinucleotides of the splice junctions (underlined in FIGS. 13A-13B) are in agreement with consensus sequences. Table 6 lists the approximate sizes of introns as determined by PCR, and their bp location in the CDNA. The introns range in size from approximately 250 bp to 4.2 kb.

Comparison of the human and mouse genes

The human and mouse genes are very similar in size and structure (FIG. 14). The introns are similar in size, and identical in location within the coding sequence. However, the mouse cDNA is one amino acid shorter in exon 1 which causes a shift in bp numbering of the mouse cDNA (Table 6, FIGS. 13A-13B). Exon 1 was defined from the original published human cDNA, based on the presence of a translation start codon. In both human and mouse genes, the 5′ boundary of exon 1 was assigned after the isolation of several non-coding cDNA extensions that are generated by alternative splicing from this junction. Characterization of these 5′ cDNA extensions is in progress. The nucleotide sequences of the human and mouse genes are very similar within coding regions, but homology decreases dramatically in the 3′ UTR region and within introns.

Human and mouse primary amino acid sequence homology

The primary amino acid sequences of human and mouse were compared to each other, and aligned with the sequence of the MetF (MTHFR) enzyme from bacteria (FIG. 15). The human and mouse amino acid sequences are almost 90% identical. As previously observed, only the 5′ half of the mammalian sequences align with the bacterial enzyme; bacterial MTHFR has the same catalytic activity as the mammalian enzyme but lacks the regulatory region in the C-terminal domain. The murine amino acid sequence is two amino acids shorter than the human sequence: one less amino acid in exon 1 and one less in exon 11.

The isolation of the human MTHFR gene and the analysis of gene structure are part of an ongoing effort to study MTHFR deficiency in homocystinuria and in multifactorial diseases. The availability of genetic structure information and of intronic sequences will help in the mutational analysis of patients suffering from MTHFR deficiency and in the characterization of the 5′ regulatory region.

Expression analysis of the 2.2 kb cDNA in a bacterial expression system resulted in a catalytically-active 7 OkDa protein (Frosst et al. 1995). A MTHFR polypeptide of this size was observed in some human tissues on Western blots, but a larger isozyme (77 kDa), corresponding to the estimated size of the porcine polypeptide, was observed in all the examined tissues. These data suggested the presence of protein isoforms for MTHFR that could be tissue-specific (Frosst et al., 1995). Since human or mouse sequences homologous to the N-terminal porcine amino acid sequences have not been identified, it is assumed that the missing sequences required to encode the larger isoform are 5′ to the available cDNA sequences. Two mRNAs for human MTHFR (approximately 7.5 and 8.5 kb) have been seen in all tissues on Northern blots (data not shown), suggesting very large UTRs. The isolation of 5′ coding sequences has been complicated by the presence of several alternatively-spliced 5′ non-coding extensions that splice into exon 1. The alternative splicing into exon 1 has been observed in both human and mouse MTHFR. The long UTRs and the alternative splicing events suggest that the regulation of this important gene may be quite complex.

Nonetheless, the available information has been critical for identification of mutations in patients with various forms of MTHFR deficiency. The mouse sequences in exons 1 and 2 have been useful in the design of antisense oligonucleotides to successfully inhibit MTHFR in mouse embryo cultures and disrupt development of the neural tube (Lanoue et al. 1997). The isolation and characterization of mouse genomic clones is essential for construction of targeting vectors to generate mouse models for MTHFR deficiency.

TABLE 6
Approximate sizes of introns, and their
locations in human and mouse MTHFR cDNA

| | Approximate | Human | Mouse |
|---|---|---|---|
| Intron | size (kb) | location¹ | location¹ |
| 1 | 1.5 | 248-249 | 245-246 |
| 2 | 0.8 | 487-488 | 484-485 |
| 3 | 4.2 | 598-599 | 595-596 |
| 4 | 0.8 | 792-793 | 789-790 |
| 5 | 0.35 | 1043-1044 | 1040-1041 |
| 6 | 0.25 | 1178-1179 | 1175-1176 |
| 7 | 0.3 | 1359-1360 | 1356-1357 |
| 8 | 1.5 | 1542-1543 | 1539-1540 |
| 9 | 1.3 | 1644-1645 | 1641-1642 |
| 10 | 0.3 | 1764-1755 | 1761-1762 |

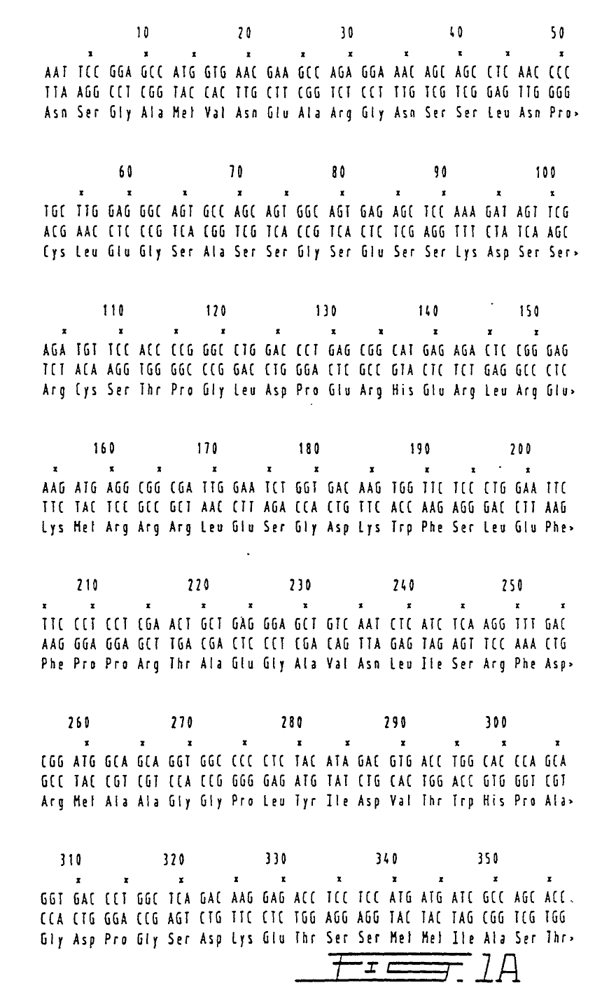

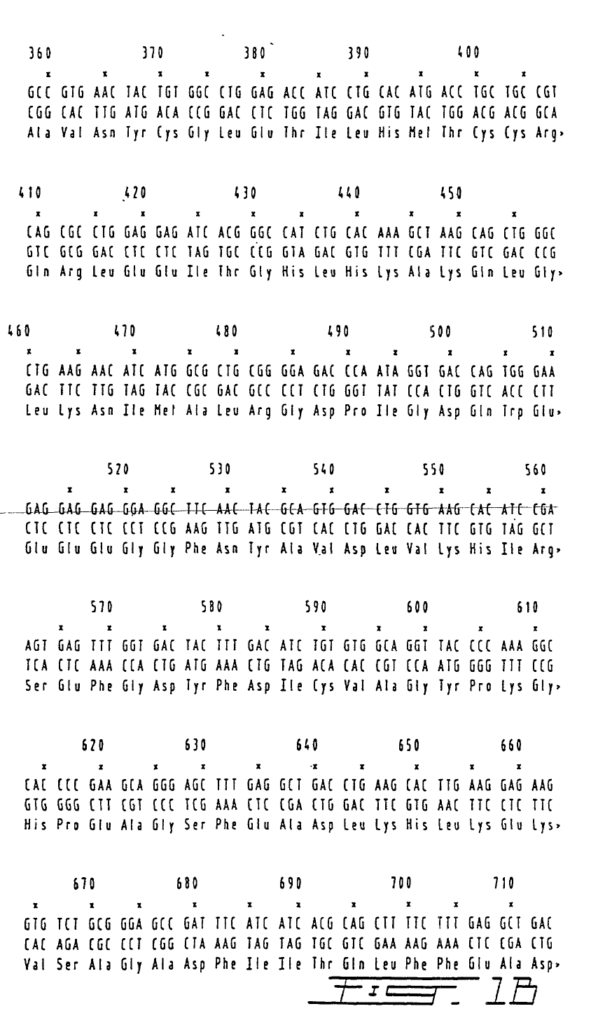

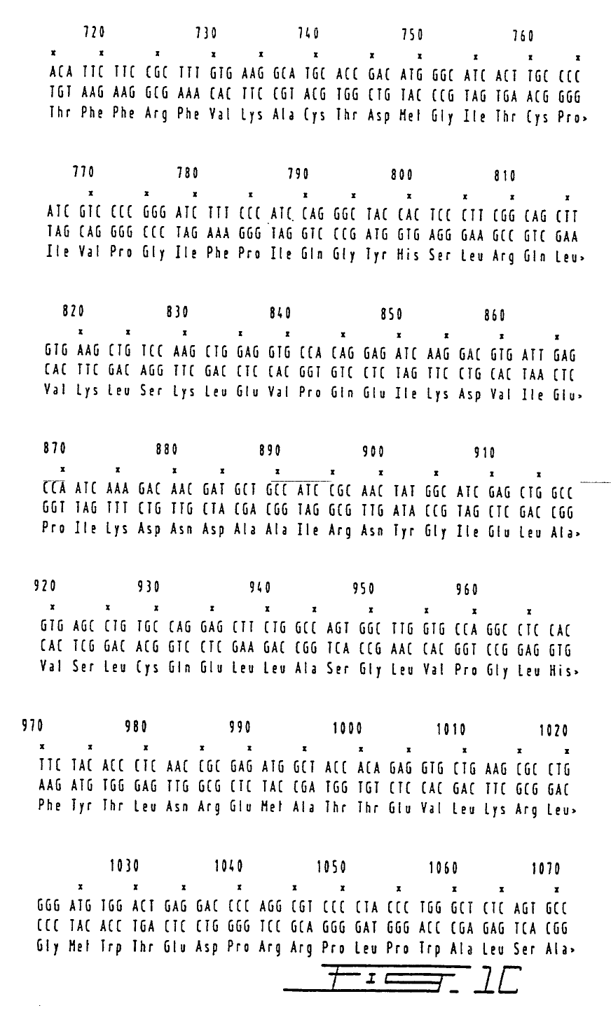

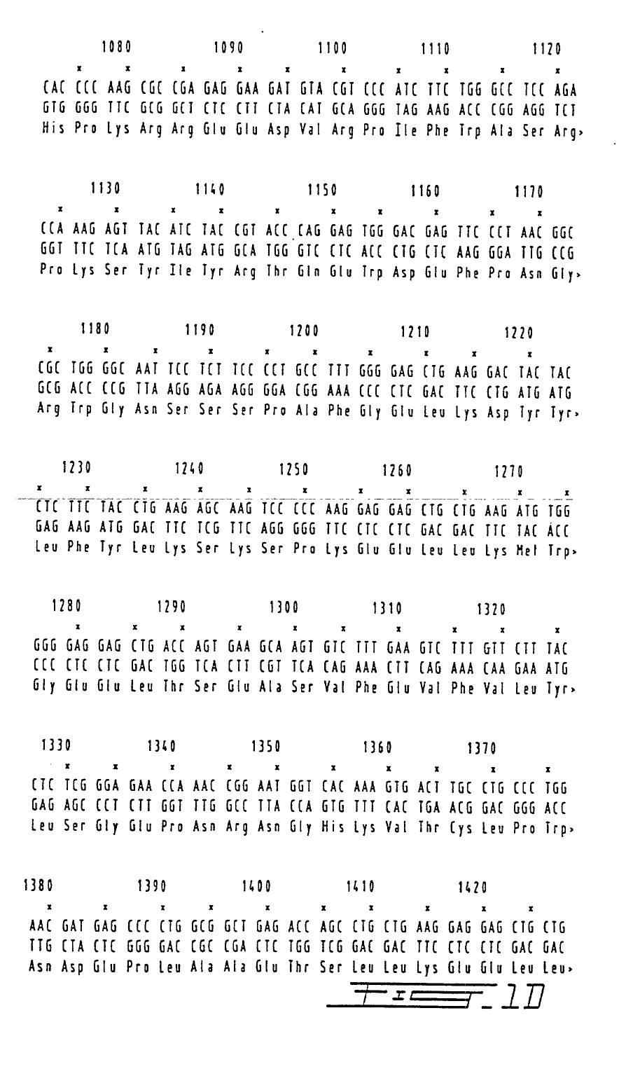

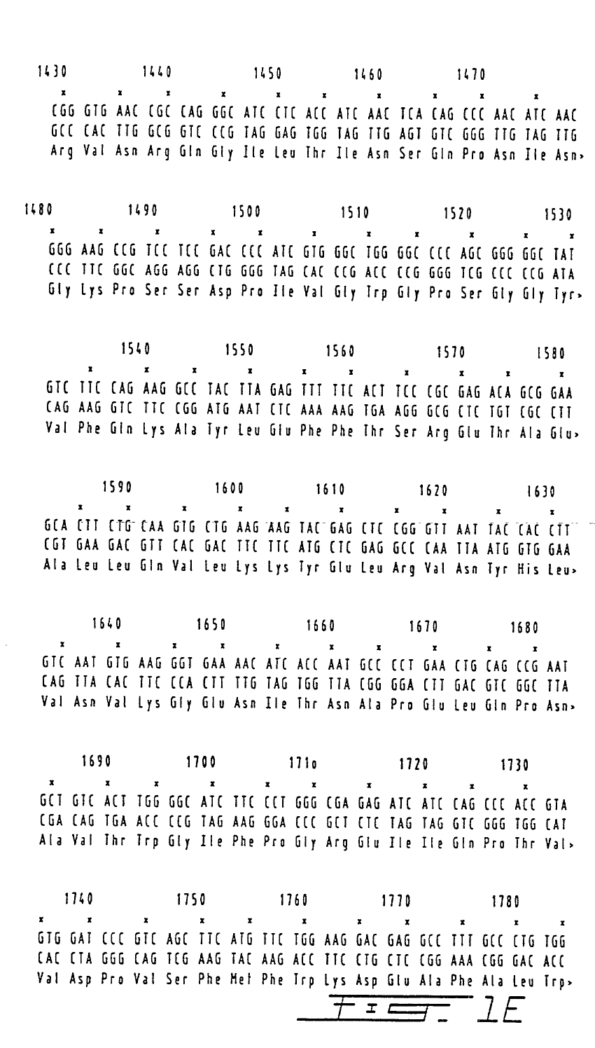

*Base pairs flanking introns, from FIGS. 1 and 2. Bpl is 12 bp upstream from the ATG, as in the original report of the cDNA sequence (Goyette et al. 1994).

Various doses of methotrexate (a drug used in treatment of cancer and arthritis, possibly other diseases) were added to colon carcinoma lines in culture. Much lower doses of methotrexate are needed to kill the lines that carry the 677C/T mutation in MTHFR, compared to lines that do not carry this mutation. The IC50 (concentration needed to kill half the cells) is approximately 20 nM for lines with the mutation and approximately 150 nM for lines without the mutation. To extrapolate to the human condition, patients with this MTHFR mutation might require lower doses of methotrexate for therapy, or might be subject to methotrexate toxicity at high doses.

TABLE 7
Summary of BMD analysis
Entire cohort: Values are means +/− SE

| | Genotype 1/1 | Genotype 1/2 | Genotype 2/2 |
|---|---|---|---|
| Spinal Z score | −1.06 (0.193) | −1.25 (0.176) | −1.86 (0.238) |
| Femoral neck Z score | −0.69 (0.134) | −0.78 (0.126) | −0.87 (0.228) |
| Trochanter Z score | −0.60 (0.142) | −0.52 (0.134) | −1.15 (0.249) |
| Ward's triangle | −0.67 (0.147) | −0.80 (0.139) | −0.96 (0.257) |
| Z score | | | |

Table 7 indicates the values for bone mineral density in a group of individuals who were examined for the MTHFR C677T mutation.
Genotype 1/1 = normal C/C
Genotype 1/2 = camers C/T
Genotype 2/2 = homozygons mutant T/T

As seen on Table 7, the lower the score, the lower the bone mineral density and therefore the higher the risk for osteoporosis. The results suggest that the homozygons mutant genotype (2/2) is asssociated with lower bone mineral density and therefore higher risk of osteoporosis.

Inhibition of MTHFR Expression for the Prevention or Treatment of Cancer

Cancer is one of the leading causes of death in the United States. Some cancers, such as melanoma, kidney cell cancer, pancreatic cancer, and brain cancer, respond poorly to chemotherapy and are termed “resistant.” Other cancers (e.g., breast cancer, small-cell lung cancer, and leukemia) may have initial favorable responses to chemotherapy but later develop resistance after repeated chemotherapy treatments. In addition, multidrug-resistance genes found in some cancer cells enable the cells to pump out drugs, rendering the cancers resistant to multiple classes of drugs. Both radiation and chemotherapy used to destroy cancerous cells also affect normal cells, resulting in a variety of possible side-effects, such as nausea, vomiting, low blood cell counts, increased risk of infection, hair loss, and ulcers in mucous membranes.

Because of the severity of these side-effects, improved cancer treatments are needed that produce fewer or less severe adverse reactions. In addition, improved therapies are needed that result in a higher incidence of remissions and longer lengths of remissions. Preferably, the therapies would prevent the initial occurrence of a cancer or prevent the recurrence of a cancer.

We have discovered that transfection of an MTHFR antisense oligonucleotide significantly decreased the viability of cancerous cells but did not decrease the viability of healthy cells that do not express functional MTHFR. In particular, a twenty base pair MTHFR antisense oligonucleotide was designed to bind exon 5. This region of exon 5 was chosen because a BLAST search of the human expressed sequence (EST) database indicated that this sequence did not have significant identity to any other reported EST in humans. In addition, no sequence variations have been reported for this MTHFR exon, suggesting that an antisense oligonucleotide to exon 5 may bind all MTHFR alleles. Transfection of this MTHFR antisense oligonucleotide significantly reduced the number of viable cells from the following cancer cell lines: SW620 colon carcinoma (ATCC Accession No. CCL-227), LOVO colon carcinoma (ATCC Accession No. CCL 229), BEC 2 neuroblastoma (ATCC Accession No. CRL 2268), SK-N-F1 neuroblastoma (ATCC Accession No. CRL 2142), MCF7 breast cancer (ATCC Accession No. HTB 2), SKBr3 breast cancer (ATCC Accession No. HTB 30), and U87-lacZ glioma cell lines (Li et al., Clin Cancer Res.5(3):637-642, 1999). In contrast, administration of the MTHFR antisense oligonucleotide to the non-cancerous WG1554 fibroblast cell line which does not produce MTHFR did not affect cell viability, suggesting that the MTHFR antisense oligonucleotide specifically inhibits MTHFR expression. In addition, Western blot analysis confirmed that the antisense oligonucleotide reduced the level of MTHFR protein but not the level of an actin control.

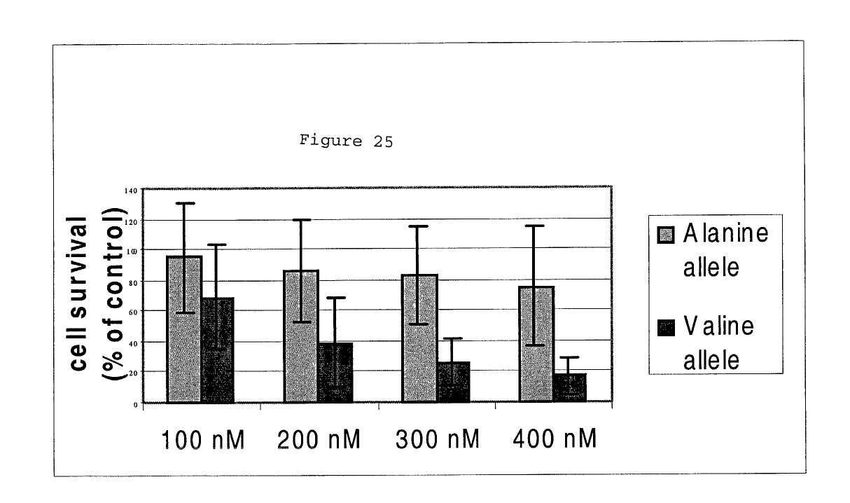

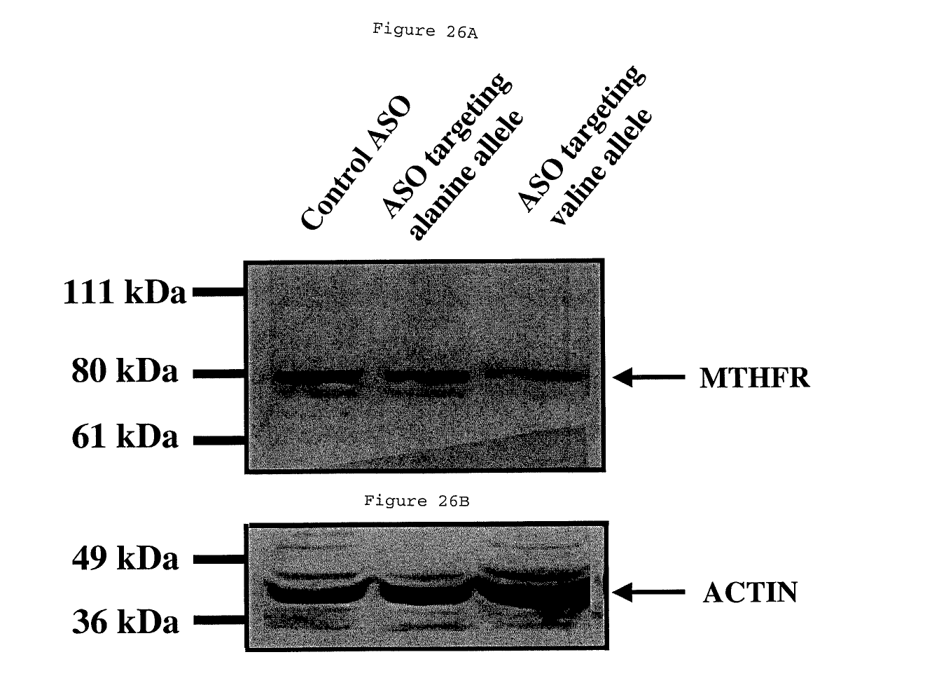

Two allele-specific MTHFR antisense oligonucleotides were also designed to bind either an MTHFR allele containing cytosine at nucleotide position 677 (valine allele) or an MTHFR allele containing thymidine at position 677 (alanine allele). These oligonucleotides were transfected into the SW620 colon carcinoma cell line, which is homozygous for thymidine at position 677. Cell viability and MTHFR protein levels were dramatically decreased following transfection with the antisense oligonucleotide designed to bind 677T (FIGS. 25 and 26). In contrast, the survival rate and level of MTHFR protein expression for cells transfected with the antisense oligonucleotide designed to bind 677C was similar to that of cells transfected with a control oligonucleotide containing six base pair mismatches. These results indicate that the allele-specific MTHFR oligonucleotides their target MTHFR allele (i.e., either the valine or alanine allele) but not both alleles.

While not meant to limit the invention to any particular theory, the inhibition of MTHFR protein expression by the MTHFR antisense oligonucleotide is thought to result in a reduced level of methionine, which is required for tumor cell growth. These results suggest that other antisense oligonucleotides or other compounds that reduce the level of MTHFR mRNA, protein, or enzymatic activity may be generally useful in the prevention, stabilization, or treatment of cancer.

For the design of additional MTHFR antisense oligonucleotides, other regions of MTHFR that have low or no sequence identity to other human ESTs may be identified by performing BLAST searches on various regions of MTHFR with the human EST database. Preferred antisense oligonucleotides do not contain three or four contiguous nucleotides that are cytosine or guanine because such regions may result in the formation of secondary or tertiary structures that reduce the affinity of the oligonucleotides for MTHFR nucleic acids. In addition, it is desirable to design antisense oligonucleotides that contain one or more single-stranded regions that are not involved in intramolecular bonding, such as base pairing. These single-stranded regions may have greater affinity for MTHFR nucleic acids than double-stranded regions. Other preferred antisense oligonucleotides include those that contain a loop or bulge region consisting of at least 10 nucleotides. Preferably, at least 1, 2, 3, 4, or 5 nucleotides at the 5′ and 3′ termini of the oligonucleotide do not participate in intramolecular interactions (Patzel et al., Nucleic Acid Research 27:4328-4334, 1999).

This present invention provides a number of advantages. For example, the methods described herein may be generally applied to the treatment, stabilization, or prevention of various types of cancer. Multiple MTHFR antisense oligonucleotides may be administered to the same subject to minimize the development of resistance to this therapy. Moreover, combination therapy involving a compound that inhibits the transcription, translation, or enzymatic activity of MTHFR and another chemotherapeutic agent or radiation therapy may be more effective than individual therapies. In addition, this combination therapy may allow smaller doses of the other chemotherapeutic agent or radiation to be used, resulting in less severe or a lower incidence of adverse side-effects.

These results are further described below.

Decreased Cell Viability in Cancer Cells after Transfection of an MTHFR Antisense Oligonucleotide

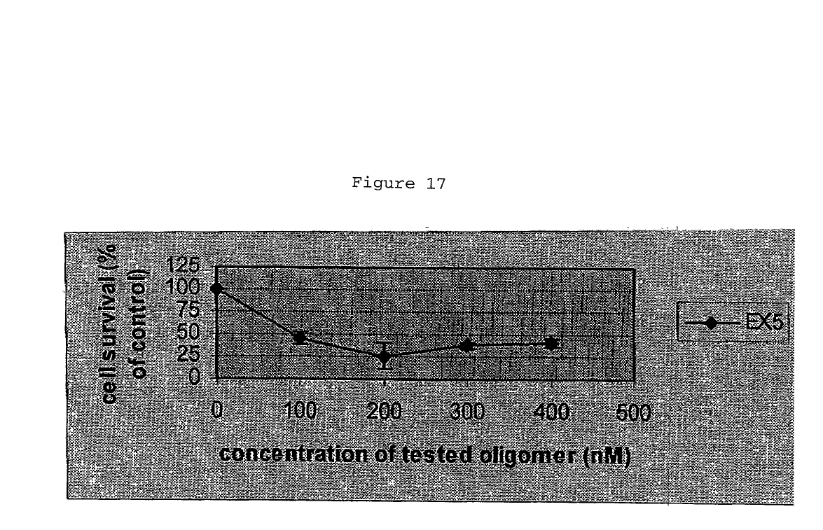

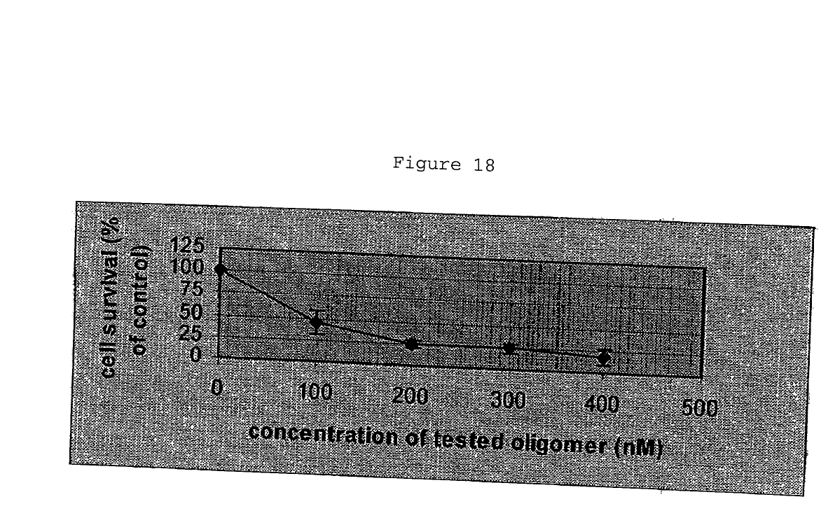

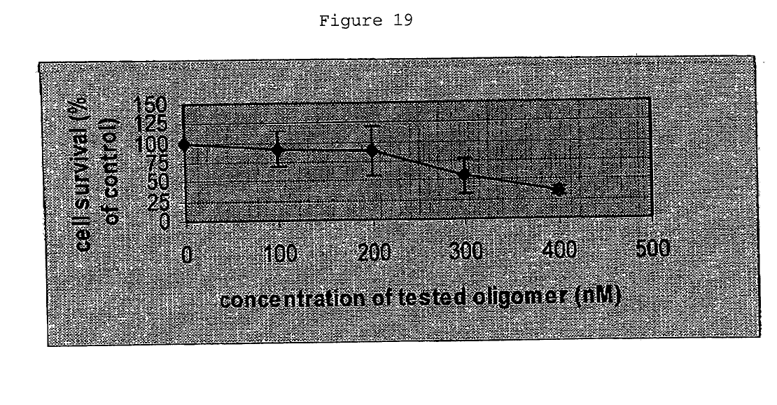

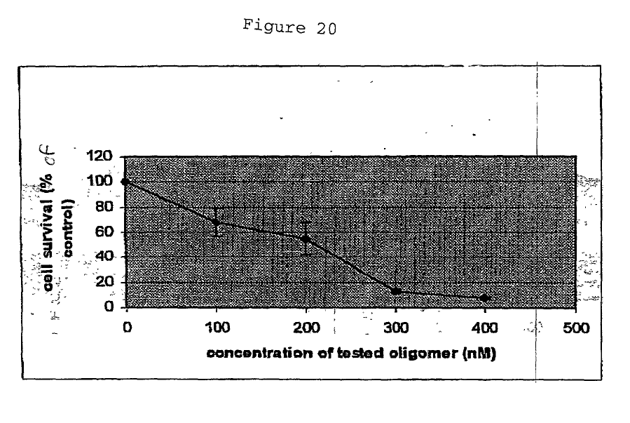

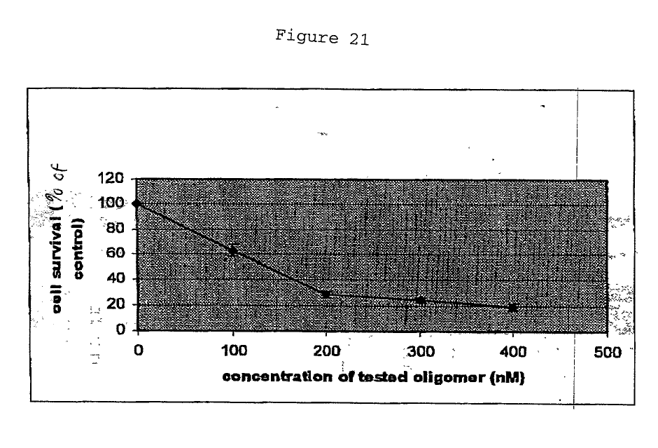

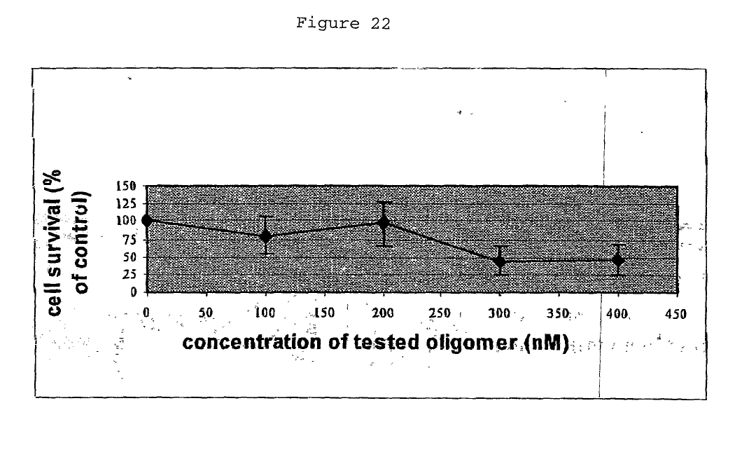

For assays to determine the effect of an MTHFR antisense oligonucleotide on cell viability, an antisense oligonucleotide (phosphorothioate 5′-AGCTGCCGAAGGGAGTGGTA-3′) designed to bind exon 5 of MTHFR, a control oligonucleotide with six base pair mismatches (phosphorothioate 5′-TGCTGTCGGAGCGATAGGTC-3′), and a control oligonucleotide with a scrambled sequence (phosphorothioate 5′-GTGACGTAGGACAGCGATGG-3′) were synthesized using standard solid-phase DNA synthesis procedures. These oligonucleotides were synthesized as phosphorothioate oligonucleotides in which one of the non-bridging phosphoryl oxygens of each nucleotide was replaced with sulfur. This modification dramatically improves nuclease stability and pharmacokinetics in vitro and in vivo. Prior to the transfection of tumor cells with these phosphorothioate oligonucleotides, the cells were plated at 50-60% confluence in 6-well plates or 10 cm tissue culture dishes and incubated overnight at 37° C. and 5% CO₂. The next day, the cells were washed with OPTI-MEM I Reduced Serum Media (Gibco, BRL) and treated with the indicated concentration of the MTHR antisense oligonucleotide or one of the control oligonucleotides (FIGS. 16-22) and 12 ug/ml Lipofectin Reagent (Gibco-BRL) in OPTI-MEM I Reduced Serum Media. For the U87-lacZ glioma cell line, the oligonucleotide with six base pair mismatches was used as the control oligonucleotide; for all the other cell lines, the scrambled oligonucleotide was used as the control oligonucleotide. After a five hour incubation at 37° C. and 5% CO₂ to allow transfection of the oligonucleotide into the cells, the OPTI-MEM I Reduced Serum Media containing the oligonucleotide and Lipofectin was replaced with MEM supplemented with 5% FBS and 5% calf serum. The next day the transfection protocol was repeated. For the second transfection, the cells were washed with Opti-MEM I media and incubated for five hours with the MTHFR antisense or control oligonucleotide and Lipofectin reagent, as described above. Then, the media was replaced with MEM supplemented with 5% FBS and 5% calf serum. The following day, a third transfection was performed as described for the second transfection. After this transfection, the cells were allowed to grow in the supplemented MEM media for two to four days. Then, the number of cells attached to the tissue culture dish was determined using a calorimetric cell protein assay kit, according to the manufacturer's protocol (FluoroReporter® Colorimetric Cell Protein Assay Kit F-2961 from Molecular Probes, Eugene Oreg.). This kit contains the anionic xanthene dye, sulforhadamine B, that forms an electrostatically stabilized complex with basic amino acid residues under moderately acidic conditions. This protein-dye complex was detected spectrophotometrically after removal of unbound die from TCA-fixed cells.

To test the effect of allele-specific MTHFR antisense oligonucleotides on tumor cell viability, an antisense oligonucleotide targeting an MTHFR allele with cytosine at nucleotide position 677 (phosphorothioate 5′-GATGAAATCGGCTCCCGCAG-3′), an antisense oligonucleotide targeting an allele with thymidine at this position (phosphorothioate 5′-GATGAAATCGACTCCCGCAG-3′), and a control oligonucleotide containing six base pair mismatches (phosphorothioate 5′-AACGATAGCGTCTCCCGCAT-3′) were synthesized as described above. SW620 colon carcinoma cells were subjected to three rounds of transfection with one of these oligonucleotides and allowed to recover for three days, as described for the non allele-specific antisense oligonucleotide to exon 5 of MTHFR.

Failure of an MTHFR Antisense Oligonucleotide to Reduce Cell Viability of Non-cancerous Cells

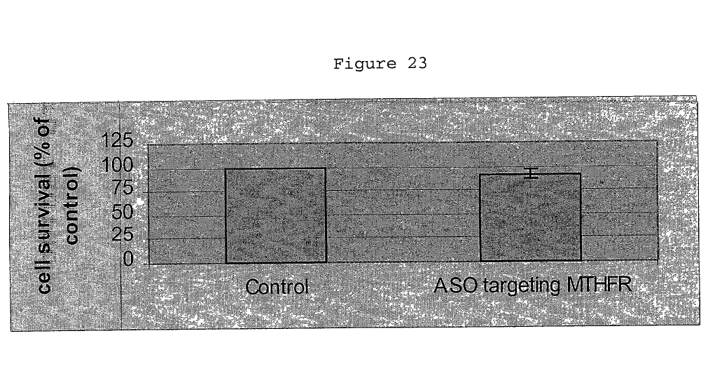

To determine the effect of an MTHFR antisense oligonucleotide on non-cancerous cells, the human diploid fibroblast cell line WG1554, which carries 2 nonsense mutations for MTHFR and thus does not produce functional MTHFR protein, was also tested in the above transfection assay (Goyette et al., Nat. Genet.7(2):195-200, 1994). In particular, the cells were subjected to three rounds of transfection with 400 nM of the antisense oligonucleotide designed to bind exon 5 of MTHFR or the control oligonucleotide and allowed to recover for three days. In contrast to the previous results with tumor cell lines, there was no difference in cell survival between WG1554 cells treated with the MTHFR antisense or control oligonucleotide (FIG. 23). This result indicates that the MTHFR antisense oligonucleotide is more toxic to cancerous cells than non-cancerous cells that do not express MTHFR. Thus, MTHFR antisense oligonucleotides may produce few adverse side-effects if administered to human subjects.

Specific Inhibition of MTHFR Protein Expression by an MTHFR Antisense Oligonucleotide

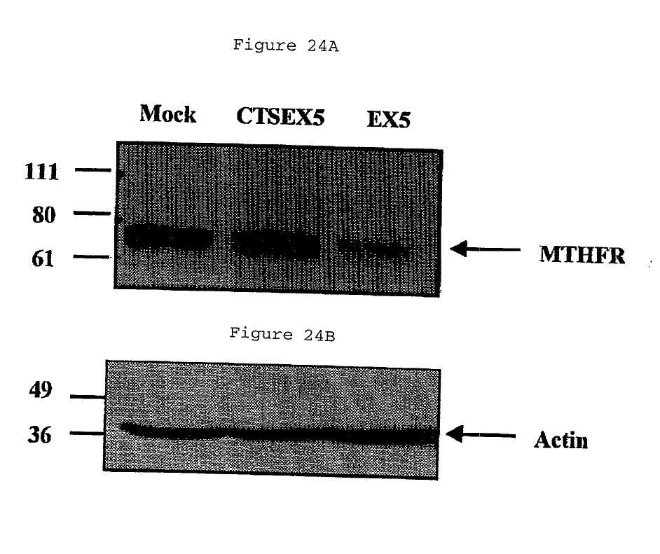

To further demonstrate that the MTHFR antisense oligonucleotides specifically inhibited MTHFR synthesis, immunologic detection of MTHFR protein was performed by Western blotting. For this assay, 600,000 cells from the SW620 colon carcinoma line were subjected to three rounds of transfection with 400 nM antisense oligonucleotide to exon 5 of MTHFR, 400 nM control scrambled oligonucleotide (phosphorothioate 5′-GTGACGTAGGACAGCGATGG-3′), or no oligonucleotide, as described above. After the last transfection, the cells were lysed using standard procedures, and the cell extracts were analyzed by Western blotting using an antibody against MTHFR and an antibody against the unrelated protein actin, as an internal control. As illustrated in FIG. 24A, the level of MTHFR protein is reduced in cells transfected with the specific antisense oligonucleotide against exon 5 (lane 3) compared to mock-transfected cells (lane 1) or cells transfected with the control mismatched oligonucleotide (lane 2). In contrast, the level of actin was not inhibited by these oligonucleotides, indicating that they do not globally inhibit protein synthesis (FIG. 24B).

To determine the effect of the allele-specific MTHFR antisense oligonucleotides on MTHFR expression, the above assay was repeated using one 24 hour transfection of the SW620 colon carcinoma cells with one of the allele-specific oligonucleotides or the corresponding control nucleotide. As illustrated in FIG. 26, MTHFR protein expression was drastically reduced by administration of the antisense oligonucleotide to the valine allele (677T). In contrast, negligible inhibition was observed following administration of the antisense oligonucleotide to the alanine allele (677C).

In Vivo Animal Model for Study of MTHFR Antisense Oligonucleotides

For in vivo testing of MTHFR antisense oligonucleotides, the previously described nude mouse cancer model may be used (Bufalo et al., British J. Canc. 74:387-393, 1996; Dean et al., (1996) Cancer Research 56:3499-3507, 1996; Hasegawa et al., Int. J. Cancer 76: 812-816, 1998; Narayanan, In Vivo 8: 787-794, 1994). Briefly, cancer cells are injected subcutaneously into the flank of a nu/nu athymic mouse. The size of the resulting tumor is measured regularly. When the tumor volume reaches 100 mm³-200 mm³, an MTHFR antisense phosphorothioate or standard oligonucleotide is injected subcutaneously at the site of the tumor. For initial experiments, a dose of 200 μg antisense oligonucleotide is injected every other day for a period of one to two weeks. During this period, the appearance and size of the tumor is monitored. After the series of injections is completed, the mouse is sacrificed, and the tumor is removed for further analysis. After the efficacy of subcutaneous administration of the antisense oligonucleotide is determined, intravenous injections may be performed to evaluate the efficacy of various doses and dosing frequencies for systemic administration of the oligonucleotide. This information may be used to determine the appropriate dosing schedule for human clinical trials. If desired, MTHFR antisense oligonucleotides may also be tested in any other standard animal model for cancer, such as naturally-occurring or induced cancers in other mammals including rats, dogs, or monkeys.

Administration of MTHFR Antisense oligonucleotides

The MTHFR antisense oligonucleotides of the invention may be administered intravenously, parenterally, subcutaneously, intramuscularly, ophthalmicly, intraventricularly, intraperitoneally, intranasally, orally, or by any other route sufficient to provide a dose adequate to treat, stabilize, or prevent cancer. Preferably, the antisense oligonucleotide is dissolved in a saline solution and administered intravenously, as described previously (Yacyshyn et al., Gastroenterology 114(6):1133-42, 1998; Neckers et al., Methods of Molecular Medicine: Antisense Therapeutics, Agrawal, ed., Humana Press, Totawa, N.J., 1996; Agrawal, Biochimica et Biophysica Acta 1489:53-68, 1999). Examples of possible mechanisms by which antisense nucleotides administered using this method are internalized by cells include pinocytosis and podocytosis. In another preferred embodiment, the oligonucleotide is administered in combination with a liposome and protamine. For any particular subject, the specific dosage regimes should be adjusted over time according to the individual need and the professional judgment of the person administering or supervising the administration of the compositions.

The MTHFR antisense oligonucleotides may also be delivered by gene therapy using any suitable method. In one preferred method, an MTHFR antisense oligonucleotide is cloned into the genome of a recombinant virus which has been modified so that it is replication deficient and does not produce virus, such as a recombinant adenoviral, adeno-associated viral, retroviral, lentiviral, baculovirus, or herpes viral vector, using standard molecular biology techniques (see, for example, Ausubel et al. (2000), supra). After administration of the recombinant virus to cells, tissues, or organs in a mammal, the antisense oligonucleotide is inserted into the genome of the mammalian cells by the viral machinery, allowing the oligonucleotide to be expressed by these cells. Preferably, the virus is administered to cancerous cells in the mammal. The general principles for this type of therapy are known to those skilled in the art and have been reviewed in the literature (see, for example, Kohn et al., Transfusion 29:812-820, 1989).

Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

All publications mentioned in this specification are herein incorporated by reference to the same extent as if each independent publication or patent application was specifically and individually indicated to be incorporated by reference.

1. A method of treating, stabilizing, or preventing cancer in a mammal, said method comprising administering to or expressing in said mammal a non allele-specific antisense nucleic acid that binds an MTHFR nucleic acid and inhibits MTHFR protein expression in said mammal, said inhibition being sufficient to treat, stabilize, or prevent cancer in said mammal.

2. The method of claim 1, wherein said cancer is a breast carcinoma, colon carcinoma, colorectal carcinoma, neuroblastoma, or glioma.

3. The method of claim 1, wherein said antisense nucleic acid binds exon 5 of said MTHFR nucleic acid.

4. The method of claim 1, wherein said antisense nucleic acid is a phosphorothioate nucleic acid.

5. The method of claim 1, wherein said antisense nucleic acid is contained in a recombinant viral vector.

6. The method of claim 1, wherein said antisense nucleic acid is administered intravenously, parenterally, subcutaneously, intramuscularly, intraventricularly, or intraperitoneally.

7. A non allele-specific antisense nucleic acid that binds an MTHFR nucleic acid and inhibits MTHFR protein expression in a mammal, said inhibition being sufficient to treat, stabilize, or prevent cancer in said mammal.

8. The antisense nucleic acid of claim 7, wherein said cancer is a breast carcinoma, colon carcinoma, colorectal carcinoma, neuroblastoma, or glioma.

9. The antisense nucleic acid of claim 7, wherein said antisense nucleic acid binds exon 5 of said MTHFR nucleic acid.

10. The antisense nucleic acid of claim 7, wherein said antisense nucleic acid is a phosphorothioate nucleic acid.

11. A vector comprising a nucleic acid encoding a non allele-specific antisense nucleic acid that binds an MTHFR nucleic acid and inhibits MTHFR protein expression in a mammal, said inhibition being sufficient to treat, stabilize, or prevent cancer in said mammal.


SEQUENCE LISTING (15 sequences)

SEQ ID NO: 1 - DNA, 2220 bp, Homo sapiens
  CDS (1)...(1980)
aattccggagccatggtgaacgaagccagaggaaacagcagcctcaacccctgcttggagggcagtgccagcagtggcagtgagagctccaaagatagttcgagatgttccaccccgggcctggaccctgagcggcatgagagactccgggagaagatgaggcggcgattggaatctggtgacaagtggttctccctggaattcttccctcctcgaactgctgagggagctgtcaatctcatctcaaggtttgaccggatggcagcaggtggccccctctacatagacgtgacctggcacccagcaggtgaccctggctcagacaaggagacctcctccatgatgatcgccagcaccgccgtgaactactgtggcctggagaccatcctgcacatgacctgctgccgtcagcgcctggaggagatcacgggccatctgcacaaagctaagcagctgggcctgaagaacatcatggcgctgcggggagacccaataggtgaccagtgggaagaggaggagggaggcttcaactacgcagtggacctggtgaagcacatccgaagtgagtttggtgactactttgacatctgtgtggcaggttaccccaaaggccaccccgaagcagggagctttgaggctgacctgaagcacttgaaggagaaggtgtctgcgggagccgatttcatcatcacgcagcttttctttgaggctgacacattcttccgctttgtgaaggcatgcaccgacatgggcatcacttgccccatcgtccccgggatctttcccatccagggctaccactcccttcggcagcttgtgaagctgtccaagctggaggtgccacaggagatcaaggacgtgattgagccaatcaaagacaacgatgctgccatccgcaactatggcatcgagctggccgtgagcctgtgccaggagcttctggccagtggcttggtgccaggcctccacttctacaccctcaaccgcgagatggctaccacagaggtgctgaagcgcctggggatgtggactgaggaccccaggcgtcccctaccctgggctctcagtgcccaccccaagcgccgagaggaagatgtacgtcccatcttctgggcctccagaccaaagagttacatctaccgtacccaggagtgggacgagttccctaacggccgctggggcaattcctcttcccctgcctttggggagctgaaggactactacctcttctacctgaagagcaagtcccccaaggaggagctgctgaagatgtggggggaggagctgaccagtgaagcaagtgtctttgaagtctttgttctttacctctcgggagaaccaaaccggaatggtcacaaagtgacttgcctgccctggaacgatgagcccctggcggctgagaccagcctgctgaaggaggagctgctgcgggtgaaccgccagggcatcctcaccatcaactcacagcccaacatcaacgggaagccgtcctccgaccccatcgtgggctggggccccagcgggggctatgtcttccagaaggcctacttagagtttttcacttcccgcgagacagcggaagcacttctgcaagtgctgaagaagtacgagctccgggttaattaccaccttgtcaatgtgaagggtgaaaacatcaccaatgcccctgaactgcagccgaatgctgtcacttggggcatcttccctgggcgagagatcatccagcccaccgtagtggatcccgtcagcttcatgttctggaaggacgaggcctttgccctgtggattgagcggtggggaaagctgtatgaggaggagtccccgtcccgcaccatcatccagtacatccacgacaactacttcctggtcaacctggtggacaatgacttcccactggacaactgcctctggcaggtggtggaagacacattggagcttctcaacaggcccacccagaatgcgagagaaacggaggctccatgaccctgcgtcctgacgccctgcgttggagccactcctgtcccgccttcctcctccacagtgctgcttctcttgggaactccactctccttcgtgtctctcccaccccggcctccactcccccacctgacaatggcagctagactggagtgaggcttccaggctcttcctggacctgagtcggccccacatgggaacctagtactctctgctctaaaaaaaaaaaaaaaaaaggaattc

SEQ ID NO: 2 - protein, 660 aa, Homo sapiens
NSGAMVNEARGNSSLNPCLEGSASSGSESSKDSSRCSTPGLDPERHERLREKMRRRLESGDKWFSLEFFPPRTAEGAVNLISRFDRMAAGGPLYIDVTWHPAGDPGSDKETSSMMIASTAVNYCGLETILHMTCCRQRLEEITGHLHKAKQLGLKNIMALRGDPIGDQWEEEEGGFNYAVDLVKHIRSEFGDYFDICVAGYPKGHPEAGSFEADLKHLKEKVSAGADFIITQLFFEADTFFRFVKACTDMGITCPIVPGIFPIQGYHSLRQLVKLSKLEVPQEIKDVIEPIKDNDAAIRNYGIELAVSLCQELLASGLVPGLHFYTLNREMATTEVLKRLGMWTEDPRRPLPWALSAHPKRREEDVRPIFWASRPKSYIYRTQEWDEFPNGRWGNSSSPAFGELKDYYLFYLKSKSPKEELLKMWGEELTSEASVFEVFVLYLSGEPNRNGHKVTCLPWNDEPLAAETSLLKEELLRVNRQGILTINSQPNINGKPSSDPIVGWGPSGGYVFQKAYLEFFTSRETAEALLQVLKKYELRVNYHLVNVKGENITNAPELQPNAVTWGIFPGREIIQPTVVDPVSFMFWKDEAFALWIERWGKLYEEESPSRTIIQYIHDNYFLVNLVDNDFPLDNCLWQVVEDTLELLNRPTQNARETEAP

SEQ ID NO: 3 - DNA, 2219 bp, Homo sapiens
  CDS (13)...(1983)
aattccggagccatggtgaacgaagccagaggaaacagcagcctcaacccctgcttggagggcagtgccagcagtggcagtgagagctccaaagatagttcgagatgttccaccccgggcctggaccctgagcggcatgagagactccgggagaagatgaggcggcgattggaatctggtgacaagtggttctccctggaattcttccctcctcgaactgctgagggagctgtcaatctcatctcaaggtttgaccggatggcagcaggtggccccctctacatagacgtgacctggcacccagcaggtgaccctggctcagacaaggagacctcctccatgatgatcgccagcaccgccgtgaactactgtggcctggagaccatcctgcacatgacctgctgccgtcagcgcctggaggagatcacgggccatctgcacaaagctaagcagctgggcctgaagaacatcatggcgctgcggggagacccaataggtgaccagtgggaagaggaggagggaggcttcaactacgcagtggacctggtgaagcacatccgaagtgagtttggtgactactttgacatctgtgtggcaggttaccccaaaggccaccccgaagcagggagctttgaggctgacctgaagcacttgaaggagaaggtgtctgcgggagccgatttcatcatcacgcagcttttctttgaggctgacacattcttccgctttgtgaaggcatgcaccgacatgggcatcacttgccccatcgtccccgggatctttcccatccagggctaccactcccttcggcagcttgtgaagctgtccaagctggaggtgccacaggagatcaaggacgtgattgagccaatcaaagacaacgatgctgccatccgcaactatggcatcgagctggccgtgagcctgtgccaggagcttctggccagtggcttggtgccaggcctccacttctacaccctcaaccgcgagatggctaccacagaggtgctgaagcgcctggggatgtggactgaggaccccaggcgtcccctaccctgggctctcagtgcccaccccaagcgccgagaggaagatgtacgtcccatcttctgggcctccagaccaaagagttacatctaccgtacccaggagtgggacgagttccctaacggccgctggggcaattcctcttcccctgcctttggggagctgaaggactactacctcttctacctgaagagcaagtcccccaaggaggagctgctgaagatgtggggggaggagctgaccagtgaagcaagtgtctttgaagtctttgttctttacctctcgggagaaccaaaccggaatggtcacaaagtgacttgcctgccctggaacgatgagcccctggcggctgagaccagcctgctgaaggaggagctgctgcgggtgaaccgccagggcatcctcaccatcaactcacagcccaacatcaacgggaagccgtcctccgaccccatcgtgggctggggccccagcgggggctatgtcttccagaaggcctacttagagtttttcacttcccgcgagacagcggaagcacttctgcaagtgctgaagaagtacgagctccgggttaattaccaccttgtcaatgtgaagggtgaaaacatcaccaatgcccctgaactgcagccgaatgctgtcacttggggcatcttccctgggcgagagatcatccagcccaccgtagtggatcccgtcagcttcatgttctggaaggacgaggcctttgccctgtggattgagcggtggggaaagctgtatgaggaggagtccccgtcccgcaccatcatccagtacatccacgacaactacttcctggtcaacctggtggacaatgacttcccactggacaactgcctctggcaggtggtggaagacacattggagcttctcaacaggcccacccagaatgcgagagaaacggaggctccatgaccctgcgtcctgacgccctgcgttggagccactcctgtcccgccttcctcctccacagtgctgcttctcttgggaactccactctccttcgtgtctctcccaccccggcctccactcccccacctgacaatggcagctagactggagtgaggcttccaggctcttcctggacctgagtcggccccacatgggaacctagtactctctgctctaaaaaaaaaaaaaaaaaaggaatt

SEQ ID NO: 4 - protein, 656 aa, Homo sapiens
MVNEARGNSSLNPCLEGSASSGSESSKDSSRCSTPGLDPERHERLREKMRRRLESGDKWFSLEFFPPRTAEGAVNLISRFDRMAAGGPLYIDVTWHPAGDPGSDKETSSMMIASTAVNYCGLETILHMTCCRQRLEEITGHLHKAKQLGLKNIMALRGDPIGDQWEEEEGGFNYAVDLVKHIRSEFGDYFDICVAGYPKGHPEAGSFEADLKHLKEKVSAGADFIITQLFFEADTFFRFVKACTDMGITCPIVPGIFPIQGYHSLRQLVKLSKLEVPQEIKDVIEPIKDNDAAIRNYGIELAVSLCQELLASGLVPGLHFYTLNREMATTEVLKRLGMWTEDPRRPLPWALSAHPKRREEDVRPIFWASRPKSYIYRTQEWDEFPNGRWGNSSSPAFGELKDYYLFYLKSKSPKEELLKMWGEELTSEASVFEVFVLYLSGEPNRNGHKVTCLPWNDEPLAAETSLLKEELLRVNRQGILTINSQPNINGKPSSDPIVGWGPSGGYVFQKAYLEFFTSRETAEALLQVLKKYELRVNYHLVNVKGENITNAPELQPNAVTWGIFPGREIIQPTVVDPVSFMFWKDEAFALWIERWGKLYEEESPSRTIIQYIHDNYFLVNLVDNDFPLDNCLWQVVEDTLELLNRPTQNARETEAP

SEQ ID NO: 5 - DNA, 22 bp, Artificial Sequence
  Synthetic primer
agcctcaacccctgcttggagg

SEQ ID NO: 6 - DNA, 22 bp, Artificial Sequence
  Synthetic primer
tgacagtttgctccccaggcac

SEQ ID NO: 7 - DNA, 23 bp, Artificial Sequence
  Synthetic primer
tgaaggagaaggtgtctgcggga

SEQ ID NO: 8 - DNA, 21 bp, Artificial Sequence
  Synthetic primer
aggacggtgcggtgagagtgg

SEQ ID NO: 9 - DNA, 23 bp, Artificial Sequence
  Synthetic primer
cactgtggttggcatggatgatg

SEQ ID NO: 10 - DNA, 21 bp, Artificial Sequence
  Synthetic primer
ggctgctcttggaccctcctc

SEQ ID NO: 11 - DNA, 21 bp, Artificial Sequence
  Synthetic primer
tgcttccggctccctctagcc

SEQ ID NO: 12 - DNA, 22 bp, Artificial Sequence
  Synthetic primer
cctcccgctcccaagaacaaag

SEQ ID NO: 13 - DNA, 23 bp, Artificial Sequence
  Synthetic primer
tgaaggagaaggtgtctgcggga

SEQ ID NO: 14 - DNA, 20 bp, Artificial Sequence
  Synthetic primer
aggacggtgcggtgagagtg

SEQ ID NO: 15 - DNA, 20 bp, Artificial Sequence
  Synthetic primer
agctgccgaagggagtggta